SEQUENCE LISTING (68 sequences)

SEQ ID NO: 1 - protein, 231 aa, Homo sapiens
MNKQRGTFSEVSLAQDPKRQQRKPKGNKSSISGTEQEIFQVELNLQNPSLNHQGIDKIYDCQGLLPPPEKLTAEVLGIICIVLMATVLKTIVLIPFLEQNNFSPNTRTQKARHCGHCPEEWITYSNSCYYIGKERRTWEESLLACTSKNSSLLSIDNEEEMKFLASILPSSWIGVFRNSSHHPWVTINGLAFKHKIKDSDNAELNCAVLQVNRLKSAQCGSSMIYHCKHKL

SEQ ID NO: 2 - protein, 138 aa, Homo sapiens
IPFLEQNNFSPNTRTQKARHCGHCPEEWITYSNSCYYIGKERRTWEESLLACTSKNSSLLSIDNEEEMKFLASILPSSWIGVFRNSSHHPWVTINGLAFKHKIKDSDNAELNCAVLQVNRLKSAQCGSSMIYHCKHKL

SEQ ID NO: 3 - protein, 347 aa, Homo sapiens
DPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 4 - protein, 242 aa, Homo sapiens
DPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVH

SEQ ID NO: 5 - protein, 7 aa, synthetic construct
GFNIKDT

SEQ ID NO: 6 - protein, 6 aa, synthetic construct
DPENGY

SEQ ID NO: 7 - protein, 10 aa, synthetic construct
SRTLFWYFDV

SEQ ID NO: 8 - protein, 17 aa, synthetic construct
KSSQSVLYSSNQKNYLA

SEQ ID NO: 9 - protein, 7 aa, synthetic construct
WASTRES

SEQ ID NO: 10 - protein, 8 aa, synthetic construct
HQYLSSYT

SEQ ID NO: 11 - protein, 25 aa, synthetic construct
EVQLQQSGAELVKPGASVTLSCTAS

SEQ ID NO: 12 - protein, 19 aa, synthetic construct
YMHWVQQRPEQGLEWIGRI

SEQ ID NO: 13 - protein, 41 aa, synthetic construct
TKYDPNFQGKATITADTSSNTAYLQLSSLTSEDTAVYHCAR

SEQ ID NO: 14 - protein, 11 aa, synthetic construct
WGAGTTVTVSS

SEQ ID NO: 15 - protein, 23 aa, synthetic construct
NIMMTQSPSSLAVSAGEKVTMSC

SEQ ID NO: 16 - protein, 15 aa, synthetic construct
WYQQKPGQSPKLLIY

SEQ ID NO: 17 - protein, 32 aa, synthetic construct
GVPDRFTGSGSGTDFTLTITNIQAEDLAVYYC

SEQ ID NO: 18 - protein, 12 aa, synthetic construct
FGGGTKLEIKRA

SEQ ID NO: 19 - protein, 119 aa, synthetic construct
EVQLQQSGAELVKPGASVTLSCTASGFNIKDTYMHWVQQRPEQGLEWIGRIDPENGYTKYDPNFQGKATITADTSSNTAYLQLSSLTSEDTAVYHCARSRTLFWYFDVWGAGTTVTVSS

SEQ ID NO: 20 - protein, 114 aa, synthetic construct
NIMMTQSPSSLAVSAGEKVTMSCKSSQSVLYSSNQKNYLAWYQQKPGQSPKLLIYWASTRESGVPDRFTGSGSGTDFTLTITNIQAEDLAVYYCHQYLSSYTFGGGTKLEIKRA

SEQ ID NO: 21 - protein, 15 aa, synthetic construct
GGGGSGGGGSGGGGS

SEQ ID NO: 22 - protein, 20 aa, synthetic construct
GGGGSGGGGSGGGGSGGGGS

SEQ ID NO: 23 - protein, 25 aa, synthetic construct
GGGGSGGGGSGGGGSGGGGSGGGGS

SEQ ID NO: 24 - protein, 7 aa, synthetic construct
GYTFTDY

SEQ ID NO: 25 - protein, 6 aa, synthetic construct
YPYNGG

SEQ ID NO: 26 - protein, 7 aa, synthetic construct
GRPAMDY

SEQ ID NO: 27 - protein, 15 aa, synthetic construct
RASESVDNYGISFMN

SEQ ID NO: 28 - protein, 7 aa, synthetic construct
AASNQGS

SEQ ID NO: 29 - protein, 9 aa, synthetic construct
QQSKEVPWT

SEQ ID NO: 30 - protein, 25 aa, synthetic construct [sequence omitted]

SEQ ID NO: 31 - protein, 19 aa, synthetic construct
NMHWVRQAPGQGLEWIGYI

SEQ ID NO: 32 - protein, 41 aa, synthetic construct
TGYNQKFKSKATITADESTNTAYMELSSLRSEDTAVYYCAR

SEQ ID NO: 33 - protein, 11 aa, synthetic construct
WGQGTLVTVSS

SEQ ID NO: 34 - protein, 23 aa, synthetic construct
DIQMTQSPSSLSASVGDRVTITC

SEQ ID NO: 35 - protein, 15 aa, synthetic construct
WFQQKPGKAPKLLIY

SEQ ID NO: 36 - protein, 32 aa, synthetic construct
GVPSRFSGSGSGTDFTLTISSLQPDDFATYYC

SEQ ID NO: 37 - protein, 10 aa, synthetic construct
FGQGTKVEIK

SEQ ID NO: 38 - protein, 116 aa, synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 39 - protein, 111 aa, synthetic construct
DIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIK

SEQ ID NO: 40 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 41 - protein, 18 aa, synthetic construct
GSTSGSGKPGSGEGSTKG

SEQ ID NO: 42 - DNA, 357 bp, synthetic construct
  source 1..357: mol_type = other DNA
gaggtacagctgcagcagtctggagccgagctggtcaaacccggcgcttctgtcactctgagctgcaccgcatctgggttcaacattaaggacacatacatgcactgggttcagcagcgcccagagcagggactggaatggattggcagaatcgaccccgaaaacggatacacgaagtatgaccccaatttccagggaaaggcaaccatcacggctgatacttcctcaaacaccgcatatttgcaactgtcatcactgaccagtgaggatactgctgtatatcactgtgctcgcagccgtaccctgttctggtatttcgacgtctggggtgccggcacaactgttactgttagttcg

SEQ ID NO: 43 - DNA, 342 bp, synthetic construct
  source 1..342: mol_type = other DNA
aacataatgatgacccagtcgccttcttccctagccgtgagcgcaggcgaaaaggtcacaatgagttgcaaatcaagccagtccgtcctgtactcgagcaaccagaaaaattacctcgcatggtatcaacagaagcctggacaatcacctaagctgctgatatattgggcttccacacgcgagtctggtgttcccgaccgatttactgggtccgggagcggcacagactttaccttgacaatcaccaatatccaggccgaggacctagctgtatattattgtcaccagtatctttctagttatactttcggcggagggactaaactcgagattaagagagcc

SEQ ID NO: 44 - DNA, 348 bp, synthetic construct
  source 1..348: mol_type = other DNA
caggtgcaactggttcaatctggcgccgaggtaaaaaaaccaggctcatctgtaaaagtgagctgtaaggcttccggatacactttcactgactacaacatgcattgggttaggcaagcacccggacaaggactcgagtggatcgggtacatatacccttacaacgggggaacagggtacaatcagaagttcaaaagtaaggctactattacagccgatgagagcactaacaccgcctacatggagcttagcagtctgagatctgaagataccgccgtgtactattgcgcacggggcagacccgccatggattactggggccagggcaccctggttacggtgtctagt

SEQ ID NO: 45 - DNA, 333 bp, synthetic construct
  source 1..333: mol_type = other DNA
gacattcagatgactcagtcccccagctcactgtctgcttccgtgggcgaccgcgtgactattacgtgtcgcgcctctgaatcagtggacaattatggcatatccttcatgaactggttccagcagaaaccaggcaaggctcccaagctgcttatatacgcggcgtctaatcaaggcagtggtgtgccttcccgattcagtggttcagggagtgggactgatttcactctgacaatttcaagcctccagccagatgatttcgctacatactactgtcaacagtctaaggaagtgccatggacattcgggcagggtaccaaggtggagatcaag

SEQ ID NO: 46 - protein, 653 aa, synthetic construct
EVQLQQSGAELVKPGASVTLSCTASGFNIKDTYMHWVQQRPEQGLEWIGRIDPENGYTKYDPNFQGKATITADTSSNTAYLQLSSLTSEDTAVYHCARSRTLFWYFDVWGAGTTVTVSSGGGGSGGGGSGGGGSNIMMTQSPSSLAVSAGEKVTMSCKSSQSVLYSSNQKNYLAWYQQKPGQSPKLLIYWASTRESGVPDRFTGSGSGTDFTLTITNIQAEDLAVYYCHQYLSSYTFGGGTKLEIKRAGSTSGSGKPGSGEGSTKGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSTSGSGKPGSGEGSTKGQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKVDEHHHHHHHHHH

SEQ ID NO: 47 - protein, 17 aa, synthetic construct
KWVTFISLLFLFSSAYS

SEQ ID NO: 48 - protein, 10 aa, synthetic construct
HHHHHHHHHH

SEQ ID NO: 49 - DNA, 2025 bp, synthetic construct
  source 1..2025: mol_type = other DNA
gccgccaccatgaagtgggtaacctttatttcccttctttttctctttagctcggcttattccgaggtacagctgcagcagtctggagccgagctggtcaaacccggcgcttctgtcactctgagctgcaccgcatctgggttcaacattaaggacacatacatgcactgggttcagcagcgcccagagcagggactggaatggattggcagaatcgaccccgaaaacggatacacgaagtatgaccccaatttccagggaaaggcaaccatcacggctgatacttcctcaaacaccgcatatttgcaactgtcatcactgaccagtgaggatactgctgtatatcactgtgctcgcagccgtaccctgttctggtatttcgacgtctggggtgccggcacaactgttactgttagttcgggcggtggcggctctggtggtggcggtagtggcggaggtggtagcaacataatgatgacccagtcgccttcttccctagccgtgagcgcaggcgaaaaggtcacaatgagttgcaaatcaagccagtccgtcctgtactcgagcaaccagaaaaattacctcgcatggtatcaacagaagcctggacaatcacctaagctgctgatatattgggcttccacacgcgagtctggtgttcccgaccgatttactgggtccgggagcggcacagactttaccttgacaatcaccaatatccaggccgaggacctagctgtatattattgtcaccagtatctttctagttatactttcggcggagggactaaactcgagattaagagagccggcagtaccagcgggtcagggaaacctggcagtggggaaggttccacaaaaggtaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctggcagtaccagcgggtcagggaaacctggcagtggggaaggttccacaaaaggtcaggtgcaactggttcaatctggcgccgaggtaaaaaaaccaggctcatctgtaaaagtgagctgtaaggcttccggatacactttcactgactacaacatgcattgggttaggcaagcacccggacaaggactcgagtggatcgggtacatatacccttacaacgggggaacagggtacaatcagaagttcaaaagtaaggctactattacagccgatgagagcactaacaccgcctacatggagcttagcagtctgagatctgaagataccgccgtgtactattgcgcacggggcagacccgccatggattactggggccagggcaccctggttacggtgtctagtggcggaggtgggagcggtggggggggtagcgggggaggcggctctgacattcagatgactcagtcccccagctcactgtctgcttccgtgggcgaccgcgtgactattacgtgtcgcgcctctgaatcagtggacaattatggcatatccttcatgaactggttccagcagaaaccaggcaaggctcccaagctgcttatatacgcggcgtctaatcaaggcagtggtgtgccttcccgattcagtggttcagggagtgggactgatttcactctgacaatttcaagcctccagccagatgatttcgctacatactactgtcaacagtctaaggaagtgccatggacattcgggcagggtaccaaggtggagatcaaggtcgacgagcatcatcatcatcaccaccaccaccaccactga

SEQ ID NO: 50 - DNA, 51 bp, synthetic construct
  source 1..51: mol_type = other DNA
aagtgggtaacctttatttcccttctttttctctttagctcggcttattcc

SEQ ID NO: 51 - DNA, 357 bp, synthetic construct; identical to SEQ ID NO: 42
  source 1..357: mol_type = other DNA

SEQ ID NO: 52 - DNA, 45 bp, synthetic construct
  source 1..45: mol_type = other DNA
ggcggtggcggctctggtggtggcggtagtggcggaggtggtagc

SEQ ID NO: 53 - DNA, 342 bp, synthetic construct; identical to SEQ ID NO: 43
  source 1..342: mol_type = other DNA

SEQ ID NO: 54 - DNA, 54 bp, synthetic construct
  source 1..54: mol_type = other DNA
ggcagtaccagcgggtcagggaaacctggcagtggggaaggttccacaaaaggt

SEQ ID NO: 55 - DNA, 342 bp, synthetic construct
  source 1..342: mol_type = other DNA
aactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 56 - DNA, 348 bp, synthetic construct; identical to SEQ ID NO: 44
  source 1..348: mol_type = other DNA

SEQ ID NO: 57 - DNA, 45 bp, synthetic construct
  source 1..45: mol_type = other DNA
ggcggaggtgggagcggtggggggggtagcgggggaggcggctct

SEQ ID NO: 58 - DNA, 333 bp, synthetic construct; identical to SEQ ID NO: 45
  source 1..333: mol_type = other DNA

SEQ ID NO: 59 - DNA, 30 bp, synthetic construct
  source 1..30: mol_type = other DNA
catcatcatcatcaccaccaccaccaccac

SEQ ID NO: 60 - DNA, 1238 bp, synthetic construct
  source 1..1238: mol_type = other DNA
ccctcacatcacacagctgcagagatgaataaacaaagaggaaccttctcagaagtgagtctggcccaggacccaaagcggcagcaaaggaaacctaaaggcaataaaagctccatttcaggaaccgaacaggaaatattccaagtagaattaaatcttcaaaatccttccctgaatcatcaagggattgataaaatatatgactgccaaggtttactgccacctccagagaagctcactgccgaggtcctaggaatcatttgcattgtcctgatggccactgtgttaaaaacaatagttcttattcctttcctggagcagaacaatttttccccgaatacaagaacgcagaaagcacgtcattgtggccattgtcctgaggagtggattacatattccaacagttgttattacattggtaaggaaagaagaacttgggaagagagtttgctggcctgtacttcgaagaactccagtctgctttctatagataatgaagaagaaatgaaatttctggccagcattttaccttcctcatggattggtgtgtttcgtaacagcagtcatcatccatgggtgacaataaatggtttggctttcaaacataagataaaagactcagataatgctgaacttaactgtgcagtgctacaagtaaatcgacttaaatcagcccagtgtggatcttcaatgatatatcattgtaagcataagctttagaagtaaagcatttgcgtttgcagtgcatcagatacattttatatttcttaaaatagaaatattatgattgcataaatctgaaaatgaattatgttatttgctctgatacaaaaattctaaatcaattattgaaataggatgcacacaattactaaagtacagacatcctagcatttgtgtcgggctcattttgctcaacatggtatttgtggttttcagcctttctaaaagttgcatgttatgtgagtcagcttataggaagtaccaagaacagtcaaacccatggagacagaaagtagaatagtggttgccaatgtctcagggaggttgaaataggagatgaccactaattgatagaacgtttctttgtgtcgtgatgaaaactttctaaatttcagtagtggtgatggttgtaactctgcgaatatactaaacatcattgatttttaatcattttaagtgcatgaaatgtatgctttgtacatgacacttcaataaagctatccagaaaaaaaaaa

SEQ ID NO: 61 - protein, 112 aa, synthetic construct
GGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 62 - DNA, 604 bp, synthetic construct
  source 1..604: mol_type = other DNA
ccacgcgtccgcgctgcgccacatcccaccggcccttacactgtggtgtccagcagcatccggcttcatggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagcacaaaaaaaaaaaaaaaaaaaaaaaaaaaaaaaaaa

SEQ ID NO: 63 - protein, 10 aa, synthetic construct
GGGGSGGGGS

SEQ ID NO: 64 - protein, 20 aa, synthetic construct
PSGQAGAAASESLFVSNHAY

SEQ ID NO: 65 - protein, 7 aa, synthetic construct
EASGGPE

SEQ ID NO: 66 - protein, 17 aa, synthetic construct
EPKSSDKTHTSPPSPEL

SEQ ID NO: 67 - protein, 25 aa, synthetic construct
RATPSHNSHQVPSAGGPTANSGTSG

SEQ ID NO: 68 - protein, 19 aa, synthetic construct
SSGGGGSGGGGGGSSRSSL

(19) United States
(12) Patent Application Publication
(10) Pub. No.: US 2026/0193362 A1
(43) Pub. Date: Jul. 9, 2026

(54) NATURAL KILLER CELL ENGAGERS

(71) Applicants: Regents of the University of Minnesota, Minneapolis, MN (US); Bio-Techne Corporation, Minneapolis, MN (US)

(72) Inventors: Martin Felices, Minneapolis, MN (US); Jeffrey Steven Miller, Minneapolis, MN (US); Jeffrey P. Houchins, Minneapolis, MN (US); Jody Bonnevier, Minneapolis, MN (US)

(21) Appl. No.: 19/134,299

(22) PCT Filed: Nov. 27, 2023
(86) PCT No.: PCT/US2023/081141

§ 371 Date: May 30, 2025

Related U.S. Application Data
(60) Provisional application No. 63/428,983, filed on Nov. 30, 2022.

(51) Int. Cl. C07K 16/28; A61K 38/00; A61P 35/02; C07K 14/54; C12N 15/62; C12N 15/86

(52) U.S. Cl. CPC C07K 16/2851; A61K 38/00; A61P 35/02; C07K 14/5443; C07K 16/2803; C12N 15/62; C12N 15/86; C07K 2317/31; C07K 2317/56; C07K 2317/622; C07K 2319/00

(57) ABSTRACT

This document relates to methods and materials involved in treating cancer. For example, this document provides cell engagers that bind to natural killer (NK) cells and bind to cancer cells. In some cases, a cell engager provided herein can include a first antigen binding domain having the ability to bind to a NK cell Group 2 isoform C (NKG2C) polypeptide and a second antigen binding domain having the ability to bind to a polypeptide present on the surface of a cancer cell. In some cases, a mammal (e.g., a human) having cancer (e.g., a leukemia such as acute myeloid leukemia (AML)) can be administered one or more cell engagers provided herein to treat the cancer.

### CROSS-REFERENCE TO RELATED APPLICATIONS

This application claims the benefit of U.S. Patent Application Ser. No. 63/428,983, filed on Nov. 30, 2022. The disclosure of the prior application is considered part of, and is incorporated by reference in, the disclosure of this application.

### STATEMENT REGARDING FEDERAL FUNDING

This invention was made with government support under W81XWH-16-1-0380 awarded by the Medical Research and Development Command, and CA197292, and CA111412 awarded by the National Institutes of Health. The government has certain rights in the invention.

### SEQUENCE LISTING

This application contains a Sequence Listing that has been submitted electronically as an XML file named “09531-0483WO1_SL.xml.” The XML file, created on Nov. 22, 2023, is 68,000 bytes in size. The material in the XML file is hereby incorporated by reference in its entirety.

### TECHNICAL FIELD

This document relates to methods and materials involved in treating cancer. For example, this document provides cell engagers that bind to natural killer (NK) cells and bind to cancer cells. In some cases, a cell engager provided herein can include a first antigen binding domain having the ability to bind to a NK cell Group 2 isoform C (NKG2C) polypeptide and a second antigen binding domain having the ability to bind to a polypeptide present on the surface of a cancer cell. In some cases, a mammal (e.g., a human) having cancer (e.g., a leukemia such as acute myeloid leukemia (AML)) can be administered one or more cell engagers provided herein to treat the cancer.

### BACKGROUND INFORMATION

NK cells are innate lymphocytes whose main function is to survey the body for virally infected and malignant cells. Upon recognition of a target cell, sufficient activation will trigger a functional response resulting in secretion of cytokines and cytolytic molecules that initiate apoptosis of the target cell. However, many tumors maintain relatively normal levels of MHC-I and evade NK cell surveillance and elimination.

### SUMMARY

This document provides methods and materials involved in treating cancer. For example, this document provides cell engagers that bind to NK cells and bind to cancer cells. In some cases, a cell engager provided herein can include a first antigen binding domain having the ability to bind to a NKG2C polypeptide and a second antigen binding domain having the ability to bind to a polypeptide present on the surface of a cancer cell. In some cases, a mammal (e.g., a human) having cancer (e.g., a leukemia such as AML) can be administered one or more cell engagers provided herein to treat the cancer.

This document also provides cells (e.g., host cells) designed to express one or more cell engagers having the ability to bind to a NKG2C polypeptide and having the ability to bind to a polypeptide present on the surface of a cancer cell, and provides methods and materials for using such cells to treat a mammal (e.g., a human) having cancer (e.g., a leukemia such as AML).

As described herein, one or more cell engagers can be designed to have the ability to bind to a NKG2C polypeptide and the ability to bind to a polypeptide present on the surface of a cancer cell (e.g., a CD33 polypeptide). For example, a cell engager provided herein can have the ability to bind to a polypeptide comprising, consisting essentially of, or consisting of the amino acid sequence of a human NKG2C polypeptide as set forth in SEQ ID NO:1 or SEQ ID NO:2 (see, e.g., Example 2) and can have the ability to bind to a polypeptide comprising, consisting essentially of, or consisting of the amino acid sequence of a human CD33 polypeptide as set forth in SEQ ID NO:3 or SEQ ID NO:4 (see, e.g., Example 5).

In some cases, a first antigen binding domain having the ability to bind to a NKG2C polypeptide and a second antigen binding domain having the ability to bind to a polypeptide present on the surface of a cancer cell can be engineered into a cell engager such as a bi-specific killer engager (e.g., a BiKE) and/or a tri-specific killer engager to create cell engagers having the ability to target NKG2C⁺ cells (e.g., NKG2C⁺ NK cells) and direct the NK cells to a target cell (e.g., a cancer cell) to induce one or more immune responses (e.g., T cell immune responses and/or antibody-dependent cell-mediated cytotoxicity (ADCC)) against the target cells. It is noted that BiKE- and tri-specific killer engager-mediated killing can be referred to ADCC even though it is not initiated by an Fc domain in those cases when a cell engager provided herein is designed to lack Fc domains.

As also described herein, one or more cell engagers provided herein can be used to treat a mammal (e.g., a human) having cancer (e.g., a leukemia such as AML). For example, a mammal (e.g., a human) having cancer (e.g., a leukemia such as AML) can be administered a composition comprising one or more cell engagers described herein to reduce the number of cancer cells within the mammal, to induce an immune response against cancer cells within the mammal, and/or to increase the survival duration of the mammal from cancer.

In general, one aspect of this document features cell engagers comprising a first antigen binding domain and a second antigen binding domain, where the first antigen binding domain can bind to a NKG2C polypeptide, and where the second antigen binding domain can bind to a polypeptide expressed on the surface of a cancer cell. The first antigen binding domain can include a heavy chain variable domain comprising the amino acid sequences set forth in SEQ ID NO:5 (or SEQ ID NO:5 with one, two, or three amino acid additions, deletions, or substitutions), SEQ ID NO:6 (or SEQ ID NO:6 with one, two, or three amino acid additions, deletions, or substitutions), and SEQ ID NO:7 (or SEQ ID NO:7 with one, two, or three amino acid additions, deletions, or substitutions), and a light chain variable domain comprising the amino acid sequences set forth in SEQ ID NO:8 (or SEQ ID NO:8 with one, two, or three amino acid additions, deletions, or substitutions), SEQ ID NO:9 (or SEQ ID NO:9 with one, two, or three amino acid additions, deletions, or substitutions), and SEQ ID NO:10 (or SEQ ID NO:10 with one, two, or three amino acid additions, deletions, or substitutions). The first antigen binding domain can include (a) a heavy chain variable domain comprising the amino acid sequence set forth in SEQ ID NO:19 and (b) a light chain variable domain comprising the amino acid sequence set forth in SEQ ID NO:20. The first antigen binding domain can be a scFv. The polypeptide expressed on the surface of the cancer cell can be a CD33 polypeptide. The second antigen binding domain can include a heavy chain variable domain comprising the amino acid sequences set forth in SEQ ID NO:24 (or SEQ ID NO:24 with one, two, or three amino acid additions, deletions, or substitutions), SEQ ID NO:25 (or SEQ ID NO:25 with one, two, or three amino acid additions, deletions, or substitutions), and SEQ ID NO:26 (or SEQ ID NO:26 with one, two, or three amino acid additions, deletions, or substitutions), and a light chain variable domain comprising the amino acid sequences set forth in SEQ ID NO:27 (or SEQ ID NO:27 with one, two, or three amino acid additions, deletions, or substitutions), SEQ ID NO:28 (or SEQ ID NO:28 with one, two, or three amino acid additions, deletions, or substitutions), and SEQ ID NO:29 (or SEQ ID NO:29 with one, two, or three amino acid additions, deletions, or substitutions). The second antigen binding domain can include (a) a heavy chain variable domain comprising the amino acid sequence set forth in SEQ ID NO:38 and (b) a light chain variable domain comprising the amino acid sequence set forth in SEQ ID NO:39. The second antigen binding domain can be a scFv. The cell engager can include a linker located between the first antigen binding domain and the second antigen binding domain. The linker can include a linker sequence selected from GGGGSGGGGSGGGGS (SEQ ID NO:21), GGGGSGGGGSGGGGSGGGGS (SEQ ID NO:22), GGGGSGGGGSGGGGSGGGGSGGGGS (SEQ ID NO:23), GGGGSGGGGS (SEQ ID NO:63), GSTSGSGKPGSGEGSTKG (SEQ ID NO:41), PSGQAGAAASESLFVSNHAY (SEQ ID NO:64), EASGGPE (SEQ ID NO:65), EPKSSDKTHTSPPSPEL (SEQ ID NO:66), RATPSHNSHQVPSAGGPTANSGTSG (SEQ ID NO:67), and SSGGGGSGGGGGGSSRSSL (SEQ ID NO:68). The cell engager can include an IL-15 polypeptide or a biologically active fragment of the IL-15 polypeptide. The IL-15 polypeptide or the biologically active fragment of the IL-15 polypeptide can be located between the first antigen binding domain and the second antigen binding domain. The IL-15 polypeptide can be separated from the first antigen binding domain by a first linker and where the IL-15 polypeptide is separated from the second antigen binding domain by a second linker. The first linker and the second linker each can include

(SEQ ID NO: 41)
GSTSGSGKPGSGEGSTKG.

In another aspect, this document features nucleic acid constructs comprising a nucleic acid sequence encoding a cell engager comprising a first antigen binding domain and a second antigen binding domain, where the first antigen binding domain can bind to a NKG2C polypeptide, and where the second antigen binding domain can bind to a polypeptide expressed on the surface of a cancer cell. The nucleic acid can be a viral vector. The nucleic acid can be a phagemid.

In another aspect, this document features host cells comprising a nucleic acid sequence encoding a cell engager comprising a first antigen binding domain and a second antigen binding domain, where the first antigen binding domain can bind to a NKG2C polypeptide, and where the second antigen binding domain can bind to a polypeptide expressed on the surface of a cancer cell.

In another aspect, this document features composition comprising a cell engager comprising a first antigen binding domain and a second antigen binding domain, where the first antigen binding domain can bind to a NKG2C polypeptide, and where the second antigen binding domain can bind to a polypeptide expressed on the surface of a cancer cell.

In another aspect, this document features methods for treating a mammal having cancer. The methods can include, or consist essentially of, administering, to a mammal having cancer, a cell engager comprising a first antigen binding domain and a second antigen binding domain, where the first antigen binding domain can bind to a NKG2C polypeptide, and where the second antigen binding domain can bind to a polypeptide expressed on the surface of a cancer cell. The mammal can be a human. The cancer can be a CD33⁺ cancer. The cancer can be a leukemia, a lymphoma, a myelodysplastic syndrome, or systemic mastocytosis. The number of cancer cells within the mammal can be reduced following the administering step.

In another aspect, this document features methods for treating a mammal having cancer. The methods can include, or consist essentially of, (a) administering, to a mammal having cancer, a cell engager comprising a first antigen binding domain and a second antigen binding domain, where the first antigen binding domain can bind to a NKG2C polypeptide, and where the second antigen binding domain can bind to a polypeptide expressed on the surface of a cancer cell, and (b) administering, to the mammal, a population of NK cells. The mammal can be a human. The cancer can be a CD33⁺ cancer. The cancer can be a leukemia, a lymphoma, a myelodysplastic syndrome, or systemic mastocytosis. At least a portion of the NK cells can be NKG2C⁺ NK cells. The NKG2C⁺ NK cells can include nucleic acid encoding a NKG2C polypeptide under conditions where the NKG2C polypeptide is expressed. At least a portion of the NK cells can include nucleic acid encoding a DAP12 polypeptide under conditions where the DAP12 polypeptide is expressed. The number of cancer cells within the mammal can be reduced following the administering steps (a) and (b).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1C. Adaptive NK cells from cytomegalovirus (CMV) seropositive healthy donors with higher frequencies of NKG2C respond to an anti-NKG2C/IL-15/anti-CD33 killer engager (also referred to herein as a NKG2C-KE). CMV seropositive donors were divided into groups based on expression of <10% or >10% NKG2C⁺ NK cells. These populations were then incubated for 5 hours with THP1 tumor cell line and stained for degranulation by CD107a (FIG. 1A) and IFNγ production (FIG. 1B). Correlations between NKG2C frequency and degranulation or IFNγ production are shown in FIG. 1C. Graphs indicate the mean+/−standard error of the mean (SEM) analyzed using a two-way ANOVA for FIG. 1A and FIG. 1B. The correlation in FIG. 1C was analyzed using a linear regression analysis. P Values as indicated: ****p<0.0001, ***p<0.01, **p<0.01, *p<0.05.

FIGS. 2A-2G. NKG2C-KE exhibit higher function in peripheral blood mononuclear cells (PBMCs) from 6-months after transplantation in those who reactivate CMV. PBMCs from patients after hematopoietic transplant were stratified as to whether they were CMV seronegative and did not reactivate CMV or whether they reactivated CMV in the first 100 days after transplant. Respective samples were incubated with THP1 tumor targets in a 5-hour assay and stained for CD107a degranulation (FIG. 2A) and IFNγ production (FIG. 2B). Correlation of CD107a degranulation (FIG. 2C) and IFNγ production (FIG. 2D) with NKG2C expression on NK cells is shown. Representative samples of CMV reactivated and CMV seronegative staining following 7-day incubation with indicated treatment after CellTrace labeling (FIG. 2E). Compiled data of proliferated NK cells in CMV reactivated (n=9) and seronegative (n=4) patients 6 months post transplantation showing percent NKG2C (FIG. 2F) and ratio of NKG2C⁺:NKG2C⁻ (FIG. 2G). Graphs indicate the mean+/−SEM analyzed using a two-way ANOVA and mixed-effect analysis for FIG. 2A and FIG. 2B, linear regression for FIG. 2C and FIG. 2D and paired t-tests for FIG. 2F and FIG. 2G. P-values as indicated: ****p<0.0001, ***p<0.01, **p<0.01, *p<0.05.

FIGS. 3A-3E. NKG2C-KE controls tumor as effectively as 161533 TriKE, which binds to CD16 on NK cells and CD33 on tumor cells. FIG. 3A) Schematic of mouse model. NOD scid gamma (NSG) mice were injected with 750,000 HL60-Luc cells, then three days later were injected with 5 million thawed expanded NK cells. Mice received 5× weekly intraperitoneal (i.p.) injections of drug for 3 weeks, bioluminescence imaging (BLI) at day 6, 13, 20, and 27, and bleeds at day 14 and 28. FIG. 3B) Frozen expanded NK cells thawed and stained for CD16 and NKG2C. FIG. 3C) Individual bioluminescence images separated into groups from day 6, 13, 20, 27 and 34. FIG. 3D) Quantification of luminescence measured at day 0, 6, 13, 20, 27 and 34. FIG. 3E) Blood from mice at day 14 and 28 stained for CD16 and NKG2C. Graphs indicate the mean+/−SD analyzed using two-way ANOVA and repeated measures (RM) analysis was used for FIG. 3C and FIG. 3D. P-values as indicated: ****p<0.0001, ***p<0.01, **p<0.01, *p<0.05.

FIGS. 4A-4C. induced pluripotent stem cell (iPSC)-derived NK (iNK) cells genetically modified to express NKG2C. FIG. 4A) iNK cells at the final stage of differentiation were stained for CD56 and NKG2C and analyzed by flow cytometry. FIG. 4B) Quantification of % NKG2C for 5 batches of iNK at the final stage of differentiation for indicated lines. FIG. 4C) MFI of indicated iNKs showing NKG2C histograms. Graphs indicate the mean+/−SD analyzed using one-way ANOVA and RM analysis used for FIG. 4B. P-values as indicated: ****p<0.0001, ***p<0.01, **p<0.01, *p<0.05.

FIGS. 5A-5E. iPSC transduced with NKG2C and DAP12 and differentiated into NK cells (iNK) exhibit CD33 specific function with the NKG2C-KE against acute myeloid leukemia (AML) targets. Non-transduced, NKG2C transduced, and NKG2C/DAP12 transduced iNK were incubated with IL-15 or NKG2C-KE for 5 hours with THP1 (FIG. 5A and FIG. 5B) or with HL60 (FIG. 5C and FIG. 5D). NK cells were stained for CD107a degranulation (FIG. 5A and FIG. 5C) and IFNγ production (FIG. 5B and FIG. 5D) in 3 separate experiments pooled in the graphs seen. The indicated iNK lines were stained with CellTrace and incubated at 37° C. for 7 days with indicated treatments as a measure of proliferation (FIG. 5E). Graphs indicate the mean+/−SD analyzed using a two-way ANOVA and RM analysis used for FIG. 5A-5D. P-values as indicated: ****p<0.0001, ***p<0.01, **p<0.01, *p<0.05.

FIGS. 6A-6C. Rapid killing kinetics with NKG2C-KE and NKG2C/DAP12 iNK. Tumor targets stained with CellTrace for tracking were incubated in the Incucyte live imaging system with iNK and indicated treatments. FIG. 6A) Images from a representative experiment at indicated times for NKG2C-KE treated iNK. FIG. 6B) Images are taken every 30 minutes to track cell numbers over time from a representative experiment. FIG. 6C) Cell numbers over time in 5 separate experiments at representative times. Graphs indicate the mean+/−SD analyzed using a two-way ANOVA and RM analysis. P-values as indicated: ****p<0.0001, ***p<0.01, **p<0.01, *p<0.05.

FIGS. 7A-7C. NKG2C-KE and NKG2C/DAP12 iNK kill primary AML targets. Primary AML cells from 5 patients were incubated with indicated iNK for 5 hours and stained for CD107a degranulation (FIG. 7A) and IFNγ production (FIG. 7B). Primary AML cells (n=3) were stained with CellTrace and incubated for 48 hours with iNK and counted by flow cytometry (FIG. 7C). Graphs indicate the mean+/−SEM analyzed using two-way ANOVA and RM analysis used for A-D. P-values as indicated: ****p<0.0001, ***p<0.01, **p<0.01, *p<0.05.

FIG. 8. NKG2C DAP12 iNK express more DAP12. Thawed iNK, non-transduced, NKG2C, and NKG2C DAP12, at the end of two-week expansion stained for intracellular DAP12.

FIGS. 9A-9B. iNK transduced with NKG2C and DAP12 do not express adaptive NK cell phenotype. FIG. 9A) Thawed iNK and adaptive NK cells were stained intracellularly for FcεRIγ, EAT2, and PLZF. FIG. 9B) Thawed iNK were stained for NKp44, KIRs, NKG2D, and NKG2A. MFI of positive iNK indicated under gating box.

FIGS. 10A-10B. NKG2C-KE directs NK cells towards CD33⁺ cells. Healthy peripheral blood NK cells divided into <10% NKG2C⁺ and >10% NKG2C⁺ (FIG. 10A) and NKG2C DAP12 iNK (FIG. 10B) incubated with Raji (CD33⁻) or THP1(CD33⁺) and indicated treatments (no drug, rhIL-15, or NKG2C-KE), in a 5-hour assay and stained for degranulation marker CD107a.

FIG. 11. Primary AML and cell lines HL-60 and THP1 express HLA-E. Cell lines and primary AML samples were thawed and rested overnight, and were stained for HLA-E in comparison to fluorescence minus one control.

FIG. 12. Schematic of an exemplary method for using NKG2C-KE to treat a human having a cancer including CD33⁺ cancer cells, such as a CD33⁺ AML.

FIGS. 13A-13B are schematics of exemplary NKG2C-KE cell engagers designed to bind (e.g., specifically bind) to a NKG2C polypeptide (e.g., a human NKG2C polypeptide). FIG. 13A shows a NKG2C-KE cell engager designed using an anti-NKG2C scFv linked to an anti-CD33 scFv. FIG. 13B shows a NKG2C-KE cell engager designed using an anti-NKG2C scFv, linked to an IL-15 polypeptide, linked to an anti-CD33 scFv.

### DETAILED DESCRIPTION

This document provides methods and materials involved in treating cancer. For example, this document provides cell engagers that bind to NK cells and bind to cancer cells. In some cases, a cell engager provided herein can include a first antigen binding domain having the ability to bind to a NKG2C polypeptide and a second antigen binding domain having the ability to bind to a polypeptide present on the surface of a cancer cell (e.g., a CD33 polypeptide). In some cases, a mammal (e.g., a human) having cancer (e.g., a leukemia such as AML) can be administered one or more cell engagers provided herein to treat the cancer.

As described herein, a cell engager can be designed to include at least one antigen binding domain having the ability to bind to a NKG2C polypeptide (e.g., a human NKG2C polypeptide) and at least one other antigen binding domain. That at least one other antigen binding domain can have the ability to bind to any appropriate antigen expressed on the surface of a cancer cell. For example, when designing a cell engager such as a BiKE to link a NKG2C⁺ NK cell and a cancer cell, the cell engager can include an antigen binding domain having the ability to bind to a NKG2C polypeptide (e.g., a human NKG2C polypeptide) and an antigen binding domain having the ability to bind to a polypeptide expressed on the surface of a cancer cell (e.g., a CD33 polypeptide).

In some cases, cell engagers provided herein can include a first antigen binding domain that can bind (e.g., specifically bind) to a polypeptide comprising, consisting essentially of, or consisting of the amino acid set forth in SEQ ID NO:1 or SEQ ID NO:2 (see, e.g., Example 2), and a second antigen binding domain that can bind (e.g., specifically bind) to a polypeptide comprising, consisting essentially of, or consisting of the amino acid set forth in SEQ ID NO:3 or SEQ ID NO:4 (see, e.g., Example 5).

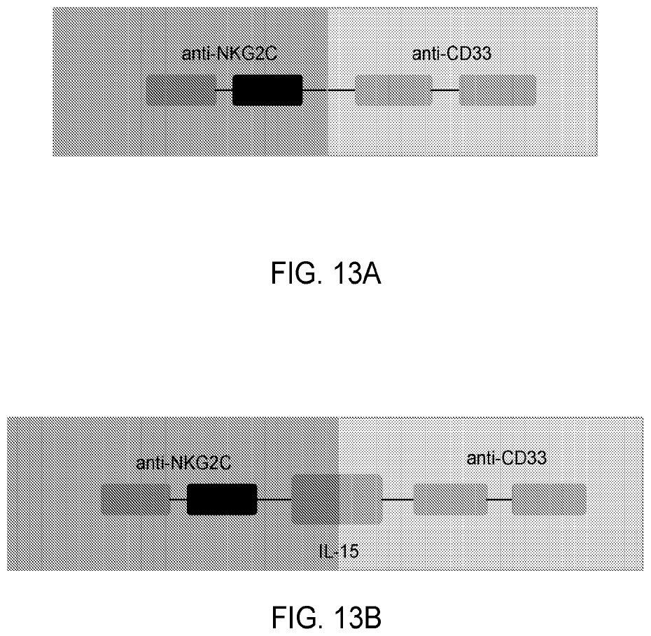

The term “cell engager” as used herein refers to a polypeptide that includes two or more antigen binding domains (e.g., two, three, or four antigen binding domains) and has the ability to link two cells together. Examples of cell engagers include, without limitation, BiTEs, BiKEs, tri-specific killer engagers, tri-specific NK engagers (TriNKETs), redirected optimized cell killing (ROCK®) engagers, diabodies, dual affinity retargeting antibodies (DARTs), and NK-cell engagers (NKCEs). In general, a cell engager provided herein can be designed to include at least one antigen binding domain having the ability to bind to a NKG2C polypeptide (e.g., a human NKG2C polypeptide) and at least one antigen binding domain having the ability to bind to a polypeptide (e.g., an antigen) expressed on the surface of a target cell (e.g., a cancer cell). In some cases, a cell engager described herein can link a NKG2C⁺ cell (e.g., a NKG2C⁺ NK cell) to another cell (e.g., a cancer cell) via the two or more antigen binding domains of the cell engager. Examples of a cell engager structures of cell engagers provided herein include, without limitation, the structures set forth in FIG. 13. In some cases, the anti-NKG2C scFv depicted in FIG. 13 can be replaced with a different antigen binding domain having the ability to bind to a polypeptide expressed on the surface of a NK cell. In some cases, the anti-CD33 scFv depicted in FIG. 13 can be replaced with a different antigen binding domain having the ability to bind to a polypeptide (e.g., an antigen) expressed on the surface of a cell (e.g., a cancer cell).

An antigen binding domain included in a cell engager provided herein can include the CDRs as described herein (e.g., as described in Table 1 and Table 2) and can be configured to be a human or humanized antigen binding domain. In some cases, an antigen binding domain included in a cell engager provided herein can include the CDRs as described herein (e.g., as described in Table 1 and Table 2) and can be configured as a scFv.

An antigen binding domain having the ability to bind to a NKG2C polypeptide (e.g., a human NKG2C polypeptide) can be any appropriate type of antigen binding domain having the ability to bind to a NKG2C polypeptide (e.g., a human NKG2C polypeptide). Examples of antigen binding domains having the ability to bind to a polypeptide expressed on the surface of a cancer cell that can be used to make a cell engager provided herein (e.g., a BiKE or a tri-specific killer engager) include, without limitation, anti-NKG2C scFvs, anti-NKG2C single-domain antibodies (sdAbs), and NKG2C-specific peptides loaded HLA-E extracellular domains.

In some cases, an antigen binding domain having the ability to bind to a NKG2C polypeptide (e.g., a human NKG2C polypeptide) that can be used to make a cell engager provided herein can include a CDR1 from a VH domain, a CDR2 from a VH domain, and a CDR3 from a VH domain, and can include a CDR1 from a VL domain, a CDR2 from a VL domain, and a CDR3 from a VL domain. For example, an antigen binding domain having the ability to bind to a NKG2C polypeptide that can be used in a cell engager provided herein can include the CDR amino acid sequences set forth below:

TABLE 1
Exemplary CDR sequences for an anti-
NKG2C antigen binding domain.

| | Sequence | SEQ ID NO |
|---|---|---|
| VH CDR1 | GFNIKDT | 5 |
| VH CDR2 | DPENGY | 6 |
| VH CDR3 | SRTLFWYFDV | 7 |
| VL CDR1 | KSSQSVLYSSNQKNYLA | 8 |
| VL CDR2 | WASTRES | 9 |
| VL CDR3 | HQYLSSYT | 10 |

In some cases, an antigen binding domain having the ability to bind to a NKG2C polypeptide (e.g., a human NKG2C polypeptide) that can be used in a cell engager provided herein including (a) a heavy chain variable domain having a CDR1 having the amino acid sequence set forth in SEQ ID NO:5 (or a variant of SEQ ID NO:5 with one, two, three, or four amino acid modifications), a CDR2 having the amino acid sequence set forth in SEQ ID NO:6 (or a variant of SEQ ID NO:6 with one, two, three, or four amino acid modifications), and a CDR3 having the amino acid sequence set forth in SEQ ID NO:7 (or a variant of SEQ ID NO:7 with one, two, three, or four amino acid modifications) and/or (b) a light chain variable domain having a CDR1 having the amino acid sequence set forth in SEQ ID NO:8 (or a variant of SEQ ID NO:8 with one, two, three, or four amino acid modifications), a CDR2 having the amino acid sequence set forth in SEQ ID NO:9 (or a variant of SEQ ID NO:9 with one, two, three, or four amino acid modifications), and a CDR3 having the amino acid sequence set forth SEQ ID NO:10 (or a variant of SEQ ID NO:10 with one, two, three, or four amino acid modifications) can include any appropriate framework regions. For example, such an antigen binding domain can include (a) a heavy chain variable domain that includes a framework region 1 having the amino acid sequence set forth in SEQ ID NO:11 (or a variant of SEQ ID NO:11 with one, two, three, four, five, six, seven, eight, nine, ten, or more amino acid modifications), a framework region 2 having the amino acid sequence set forth in SEQ ID NO:12 (or a variant of SEQ ID NO:12 with one, two, three, four, five, six, seven, eight, nine, ten, or more amino acid modifications), a framework region 3 having the amino acid sequence set forth in SEQ ID NO:13 (or a variant of SEQ ID NO:13 with one, two, three, four, five, six, seven, eight, nine, ten, or more amino acid modifications), and a framework region 4 having the amino acid sequence set forth in SEQ ID NO:14 (or a variant of SEQ ID NO:14 with one, two, three, four, five, six, seven, eight, nine, ten, or more amino acid modifications) and/or (b) a light chain variable domain that includes a framework region 1 having the amino acid sequence set forth in SEQ ID NO:15 (or a variant of SEQ ID NO:15 with one, two, three, four, five, six, seven, eight, nine, ten, or more amino acid modifications), a framework region 2 having the amino acid sequence set forth in SEQ ID NO:16 (or a variant of SEQ ID NO:16 with one, two, three, four, five, six, seven, eight, nine, ten, or more amino acid modifications), a framework region 3 having the amino acid sequence set forth in SEQ ID NO:17 (or a variant of SEQ ID NO:17 with one, two, three, four, five, six, seven, eight, nine, ten, or more amino acid modifications), and a framework region 4 having the amino acid sequence set forth in SEQ ID NO:18 (or a variant of SEQ ID NO:18 with one, two, three, four, five, six, seven, eight, nine, ten, or more amino acid modifications).

In some cases, an antigen binding domain having the ability to bind to a NKG2C polypeptide (e.g., a human NKG2C polypeptide) that can be used in a cell engager provided herein can include a heavy chain variable domain comprising SEQ ID NO:19 and a light chain variable domain comprising SEQ ID NO:20.

In some cases, an antigen binding domain having the ability to bind to a NKG2C polypeptide (e.g., a human NKG2C polypeptide) that can be used in a cell engager provided herein can include (a) a heavy chain variable domain that includes an amino acid sequence having at least 90 percent identity to the amino acid sequence set forth in SEQ ID NO:19 and/or (b) a light chain variable domain that includes an amino acid sequence having at least 90 percent identity to the amino acid sequence set forth in SEQ ID NO:20. For example, such an antigen binding domain can include (a) a heavy chain variable domain that includes an amino acid sequence having at least 90, 91, 92, 93, 94, 95, 96, 97, 98, or 99 percent identity to the amino acid sequence set forth in SEQ ID NO:19 and/or (b) a light chain variable domain that includes an amino acid sequence having at least 90, 91, 92, 93, 94, 95, 96, 97, 98, or 99 percent identity to the amino acid sequence set forth in SEQ ID NO:20. In some cases, an antigen binding domain having the ability to bind to a NKG2C polypeptide (e.g., a human NKG2C polypeptide) that can be used in a cell engager provided herein can include (a) a heavy chain variable domain that includes an amino acid sequence having at least 90 percent identity to the amino acid sequence set forth in SEQ ID NO:19, provided that the heavy chain variable domain includes the amino acid sequences set forth in SEQ ID NOs:5, 6, and 7, and/or (b) a light chain variable domain that includes an amino acid sequence having at least 90 percent identity to the amino acid sequence set forth in SEQ ID NO:20, provided that the light chain variable domain includes the amino acid sequences set forth in SEQ ID NOs:8, 9, and 10.

In some cases, an antigen binding domain having the ability to bind to a NKG2C polypeptide (e.g., a human NKG2C polypeptide) that can be used in a cell engager provided herein can include (a) a heavy chain variable domain having the amino acid sequence set forth in SEQ ID NO:19 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO:20 with one, two, three, four, five, six, seven, eight, nine, or 10 amino acid modifications (e.g., amino acid substitutions, amino acid deletions, and/or amino acid additions). For example, such an antigen binding domain can include a heavy chain variable domain having the amino acid sequence set forth in SEQ ID NO:19 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:5, 6, and 7, and can include a light chain variable domain having the amino acid sequence set forth in SEQ ID NO:20 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs:8, 9, and 10.

In some cases, an antigen binding domain having the ability to bind to a NKG2C polypeptide (e.g., a human NKG2C polypeptide) that can be used in a cell engager provided herein can include (a) a heavy chain variable domain comprising (i) a CDR1 that comprises, consists essentially of, or consists of the amino acid sequence set forth in SEQ ID NO:5, (ii) a CDR2 that comprises, consists essentially of, or consists of the amino acid sequence set forth in SEQ ID NO:6, and (iii) a CDR3 that comprises, consists essentially of, or consists of the amino acid sequence set forth in SEQ ID NO:7, and/or (b) a light chain variable domain comprising (i) a CDR1 that comprises, consists essentially of, or consists of the amino acid sequence set forth in SEQ ID NO:8, (ii) a CDR2 that comprises, consists essentially of, or consists of the amino acid sequence set forth in SEQ ID NO:9, and (iii) a CDR3 that comprises, consists essentially of, or consists of the amino acid sequence set forth in SEQ ID NO:10.

When designing an antigen binding domain to be a scFv) having a heavy chain variable domain and a light chain variable domain, the two regions can be directly connected or can be connected using any appropriate linker sequence. In some cases, a heavy chain variable domain having the CDRs of SEQ ID NOs:5-7 can be directly connected to a light chain variable domain having the CDRs of SEQ ID NOs:8-10. In some cases, a heavy chain variable domain having the CDRs of SEQ ID NOs:5-7 can be connected to a light chain variable domain having the CDRs of SEQ ID NOs:8-10 via a linker sequence. For example, an antigen binding domain targeting a NKG2C polypeptide can include a heavy chain variable domain comprising SEQ ID NO:19, followed by a linker, followed by a light chain variable domain comprising SEQ ID NO:20. In another example, an antigen binding domain targeting a NKG2C polypeptide can include a light chain variable domain comprising SEQ ID NO:20, followed by a linker, followed by a heavy chain variable domain comprising SEQ ID NO:19. A linker can be any appropriate length. For example, a linker that can be used to connect a heavy chain variable domain and a light chain variable domain can be from about 3 to about 100 (e.g., from about 3 to about 90, from about 3 to about 80, from about 3 to about 70, from about 3 to about 60, from about 3 to about 50, from about 3 to about 40, from about 3 to about 30, from about 3 to about 20, from about 3 to about 15, from about 5 to about 100, from about 10 to about 100, from about 20 to about 100, from about 30 to about 100, from about 40 to about 100, from about 50 to about 100, from about 60 to about 100, from about 70 to about 100, from about 10 to about 50, from about 10 to about 40, from about 10 to about 30, from about 10 to about 20, or from about 12 to about 17) amino acid residues in length. Examples of linker sequences that can be used to connect a heavy chain variable domain and a light chain variable domain to create an antigen binding domain include, without limitation, GGGGSGGGGSGGGGS (SEQ ID NO:21), GGGGSGGGGSGGGGSGGGGS (SEQ ID NO:22), GGGGSGGGGSGGGGSGGGGSGGGGS (SEQ ID NO:23), GGGGSGGGGS (SEQ ID NO:63), GSTSGSGKPGSGEGSTKG (SEQ ID NO:41), PSGQAGAAASESLFVSNHAY (SEQ ID NO:64), EASGGPE (SEQ ID NO:65), EPKSSDKTHTSPPSPEL (SEQ ID NO:66), RATPSHNSHQVPSAGGPTANSGTSG (SEQ ID NO:67), and SSGGGGSGGGGGGSSRSSL (SEQ ID NO:68).

An antigen binding domain having the ability to bind to a polypeptide present on the surface of a target cell can bind to a polypeptide on the surface of any appropriate type of cell. In some cases, a target cell can be a cancer cell.

An antigen binding domain having the ability to bind to a polypeptide present on the surface of a target cell (e.g., a cancer cell) can bind to any appropriate polypeptide on the surface of the target cell. In some cases, a polypeptide expressed on the surface of a cancer cell can be a tumor-associated antigen. In some cases, a polypeptide expressed on the surface of a cancer cell can be a tumor-specific antigen. Examples of polypeptides that can be present on the surface of a target cell (e.g., a cancer cell) and can be targeted by an antigen binding domain of a cell engager provided herein include, without limitation, CD33 polypeptides, B7-H3 polypeptides, PSMA polypeptides, TEM-8 polypeptides, HER2 polypeptides, mesothelin polypeptides, EPCAM polypeptides, CD133 polypeptides, CSPG4 polypeptides, CLEC12A polypeptides, CD19 polypeptides, CD22 polypeptides, ROR1 polypeptides, IGF1R polypeptides, IRA polypeptides, and PD-L1 polypeptides. Examples of antigen binding domains having the ability to bind to a polypeptide expressed on the surface of a cancer cell that can be used to make a cell engager provided herein (e.g., a BiKE or a tri-specific killer engager) include, without limitation, anti-CD33 scFvs and anti-CD33 sdAbs.

In some cases, an antigen binding domain having the ability to bind to a CD33 polypeptide (e.g., a human CD33 polypeptide) that can be used in a cell engager provided herein can include a CDR1 from a VH domain, a CDR2 from a VH domain, and a CDR3 from a VH domain, and can include a CDR1 from a VL domain, a CDR2 from a VL domain, and a CDR3 from a VL domain. For example, an antigen binding domain having the ability to bind to a CD33 polypeptide that can be used in a cell engager provided herein can include the CDR amino acid sequences set forth in Table 2.

TABLE 2
Exemplary CDR sequences for an anti-
CD33 antigen binding domain.

| | Sequence | SEQ ID NO |
|---|---|---|
| VH CDR1 | GYTFTDY | 24 |
| VH CDR2 | YPYNGG | 25 |
| VH CDR3 | GRPAMDY | 26 |
| VL CDR1 | RASESVDNYGISFMN | 27 |
| VL CDR2 | AASNQGS | 28 |
| VL CDR3 | QQSKEVPWT | 29 |

In some cases, an antigen binding domain having the ability to bind to a CD33 polypeptide (e.g., a human CD33 polypeptide) that can be used in a cell engager provided herein including (a) a heavy chain variable domain having a CDR1 having the amino acid sequence set forth in SEQ ID NO:24 (or a variant of SEQ ID NO:24 with one, two, three, or four amino acid modifications), a CDR2 having the amino acid sequence set forth in SEQ ID NO:25 (or a variant of SEQ ID NO:25 with one, two, three, or four amino acid modifications), and a CDR3 having the amino acid sequence set forth in SEQ ID NO:26 (or a variant of SEQ ID NO:26 with one, two, three, or four amino acid modifications) and/or (b) a light chain variable domain having a CDR1 having the amino acid sequence set forth in SEQ ID NO:27 (or a variant of SEQ ID NO:27 with one, two, three, or four amino acid modifications), a CDR2 having the amino acid sequence set forth in SEQ ID NO:28 (or a variant of SEQ ID NO:28 with one, two, three, or four amino acid modifications), and a CDR3 having the amino acid sequence set forth SEQ ID NO:29 (or a variant of SEQ ID NO:29 with one, two, three, or four amino acid modifications) can include any appropriate framework regions. For example, such an antigen binding domain can include (a) a heavy chain variable domain that includes a framework region 1 having the amino acid sequence set forth in SEQ ID NO:30 (or a variant of SEQ ID NO:30 with one, two, three, four, five, six, seven, eight, nine, ten, or more amino acid modifications), a framework region 2 having the amino acid sequence set forth in SEQ ID NO:31 (or a variant of SEQ ID NO:31 with one, two, three, four, five, six, seven, eight, nine, ten, or more amino acid modifications), a framework region 3 having the amino acid sequence set forth in SEQ ID NO:32 (or a variant of SEQ ID NO:32 with one, two, three, four, five, six, seven, eight, nine, ten, or more amino acid modifications), and a framework region 4 having the amino acid sequence set forth in SEQ ID NO:33 (or a variant of SEQ ID NO:33 with one, two, three, four, five, six, seven, eight, nine, ten, or more amino acid modifications) and/or (b) a light chain variable domain that includes a framework region 1 having the amino acid sequence set forth in SEQ ID NO:34 (or a variant of SEQ ID NO:34 with one, two, three, four, five, six, seven, eight, nine, ten, or more amino acid modifications), a framework region 2 having the amino acid sequence set forth in SEQ ID NO:35 (or a variant of SEQ ID NO:35 with one, two, three, four, five, six, seven, eight, nine, ten, or more amino acid modifications), a framework region 3 having the amino acid sequence set forth in SEQ ID NO:36 (or a variant of SEQ ID NO:36 with one, two, three, four, five, six, seven, eight, nine, ten, or more amino acid modifications), and a framework region 4 having the amino acid sequence set forth in SEQ ID NO:37 (or a variant of SEQ ID NO:37 with one, two, three, four, five, six, seven, eight, nine, ten, or more amino acid modifications).

In some cases, an antigen binding domain having the ability to bind to a CD33 polypeptide (e.g., a human CD33 polypeptide) that can be used in a cell engager provided herein can include a heavy chain variable domain comprising SEQ ID NO:38 and a light chain variable domain comprising SEQ ID NO:39.

In some cases, an antigen binding domain having the ability to bind to a CD33 polypeptide (e.g., a human CD33 polypeptide) that can be used in a cell engager provided herein can include (a) a heavy chain variable domain that includes an amino acid sequence having at least 90 percent identity to the amino acid sequence set forth in SEQ ID NO:38 and/or (b) a light chain variable domain that includes an amino acid sequence having at least 90 percent identity to the amino acid sequence set forth in SEQ ID NO:39. For example, such an antigen binding domain can include (a) a heavy chain variable domain that includes an amino acid sequence having at least 90, 91, 92, 93, 94, 95, 96, 97, 98, or 99 percent identity to the amino acid sequence set forth in SEQ ID NO:38 and/or (b) a light chain variable domain that includes an amino acid sequence having at least 90, 91, 92, 93, 94, 95, 96, 97, 98, or 99 percent identity to the amino acid sequence set forth in SEQ ID NO:39. In some cases, an antigen binding domain having the ability to bind to a CD33 polypeptide (e.g., a human CD33 polypeptide) that can be used in a cell engager provided herein can include (a) a heavy chain variable domain that includes an amino acid sequence having at least 90 percent identity to the amino acid sequence set forth in SEQ ID NO:38, provided that the heavy chain variable domain includes the amino acid sequences set forth in SEQ ID NOs:24, 25, and 26, and/or (b) a light chain variable domain that includes an amino acid sequence having at least 90 percent identity to the amino acid sequence set forth in SEQ ID NO:39, provided that the light chain variable domain includes the amino acid sequences set forth in SEQ ID NOs:27, 28, and 28.

In some cases, an antigen binding domain having the ability to bind to a CD33 polypeptide (e.g., a human CD33 polypeptide) that can be used in a cell engager provided herein can include (a) a heavy chain variable domain having the amino acid sequence set forth in SEQ ID NO:38 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO:39 with one, two, three, four, five, six, seven, eight, nine, or 10 amino acid modifications (e.g., amino acid substitutions, amino acid deletions, and/or amino acid additions). For example, such an antigen binding domain can include a heavy chain variable domain having the amino acid sequence set forth in SEQ ID NO:38 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:24, 25, and 26, and can include a light chain variable domain having the amino acid sequence set forth in SEQ ID NO:39 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs:27, 28, and 29.

In some cases, an antigen binding domain having the ability to bind to a CD33 polypeptide (e.g., a human CD33 polypeptide) that can be used in a cell engager provided herein can include (a) a heavy chain variable domain comprising (i) a CDR1 that comprises, consists essentially of, or consists of the amino acid sequence set forth in SEQ ID NO:24, (ii) a CDR2 that comprises, consists essentially of, or consists of the amino acid sequence set forth in SEQ ID NO:25, and (iii) a CDR3 that comprises, consists essentially of, or consists of the amino acid sequence set forth in SEQ ID NO:26, and/or (b) a light chain variable domain comprising (i) a CDR1 that comprises, consists essentially of, or consists of the amino acid sequence set forth in SEQ ID NO:27, (ii) a CDR2 that comprises, consists essentially of, or consists of the amino acid sequence set forth in SEQ ID NO:28, and (iii) a CDR3 that comprises, consists essentially of, or consists of the amino acid sequence set forth in SEQ ID NO:29.

When designing an antigen binding domain as a scFv having a heavy chain variable domain and a light chain variable domain, the two regions can be directly connected or can be connected using any appropriate linker sequence. In some cases, a heavy chain variable domain having the CDRs of SEQ ID NOs:24-26 can be directly connected to a light chain variable domain having the CDRs of SEQ ID NOs:27-29. In some cases, a heavy chain variable domain having the CDRs of SEQ ID NOs:24-26 can be connected to a light chain variable domain having the CDRs of SEQ ID NOs:27-29 via a linker sequence. For example, an antigen binding domain targeting a CD33 polypeptide can include a heavy chain variable domain comprising SEQ ID NO:38, followed by a linker, followed by a light chain variable domain comprising SEQ ID NO:39. For example, an antigen binding domain targeting a CD33 polypeptide can include a light chain variable domain comprising SEQ ID NO:39, followed by a linker, followed by a heavy chain variable domain comprising SEQ ID NO:38. A linker can be any appropriate length. For example, a linker that can be used to connect a heavy chain variable domain and a light chain variable domain can be from about 3 to about 100 (e.g., from about 3 to about 90, from about 3 to about 80, from about 3 to about 70, from about 3 to about 60, from about 3 to about 50, from about 3 to about 40, from about 3 to about 30, from about 3 to about 20, from about 3 to about 15, from about 5 to about 100, from about 10 to about 100, from about 20 to about 100, from about 30 to about 100, from about 40 to about 100, from about 50 to about 100, from about 60 to about 100, from about 70 to about 100, from about 10 to about 50, from about 10 to about 40, from about 10 to about 30, from about 10 to about 20, or from about 12 to about 17) amino acid residues in length. Examples of linker sequences that can be used to connect a heavy chain variable domain and a light chain variable domain to create an antigen binding domain include, without limitation, GGGGSGGGGSGGGGS (SEQ ID NO:21), GGGGSGGGGSGGGGSGGGGS (SEQ ID NO:22), GGGGSGGGGSGGGGSGGGGSGGGGS (SEQ ID NO:23), GGGGSGGGGS (SEQ ID NO:63), GSTSGSGKPGSGEGSTKG (SEQ ID NO:41), PSGQAGAAASESLFVSNHAY (SEQ ID NO:64), EASGGPE (SEQ ID NO:65), EPKSSDKTHTSPPSPEL (SEQ ID NO:66), RATPSHNSHQVPSAGGPTANSGTSG (SEQ ID NO:67), and SSGGGGSGGGGGGSSRSSL (SEQ ID NO:68).

In some cases, a cell engager provided herein can include one or more additional components (e.g., one or more additional polypeptides). For example, a cell engager provided herein can include a first antigen binding domain that can bind (e.g., specifically bind) to a polypeptide present on the surface of a NK cell (e.g., a NKG2C polypeptide), a second antigen binding domain that can bind (e.g., specifically bind) to a polypeptide present on the surface of a cancer cell (e.g., a CD33 polypeptide), and can include one or more additional polypeptides. Examples of additional polypeptides that can be included in a cell engager provided herein include, without limitation, polypeptides that can promote NK cell proliferation and/or survival (e.g., IL-15 polypeptides), signal polypeptides, and detectable polypeptides.

In some cases, a cell engager provided herein can be designed to include an IL-15 polypeptide. An IL-15 polypeptide can be any appropriate IL-15 polypeptide. In some cases, an IL-15 polypeptide can be a human IL-15 polypeptide (e.g., recombinant human IL-15 (rhIL-15) polypeptide). An example of an IL-15 polypeptide that can be included in a cell engager provided herein includes, without limitation, the amino acid sequence set forth in SEQ ID NO:40 (see, e.g., Example 8). In some cases, a cell engager provided herein can be designed to include a functional fragment or a variant of the amino acid sequence set forth in SEQ ID NO:40 provided that the variant or fragment maintains its basic abilities to proliferate, prime, and survive. For example, a cell engager provided herein can be designed to include an IL-15 polypeptide that comprises, consists essentially of, or consists of the amino acid sequence set forth in SEQ ID NO:40 with two or less, three or less, four or less, five or less, six or less, seven or less, eight or less, nine or less, or ten or less amino acid deletions, additions, substitutions, or combinations thereof.

In some cases, a cell engager provided herein can be designed to include a signal polypeptide. Any appropriate signal polypeptide can be used to design a cell engager described herein. Examples of signal polypeptides that can be used to make a cell engager described herein include without limitation, BLK Alb signal polypeptides, tPA signal polypeptides, BiP signal polypeptides, and CD8α signal polypeptides.

When a cell engager provided herein includes one or more additional components (e.g., one or more additional polypeptides), the additional component(s) can be located at any appropriate location within the cell engager. In some cases, a cell engager provided herein can have an IL-15 polypeptide located between a first antigen binding domain having the ability to bind to a NKG2C polypeptide and a second antigen binding domain having the ability to bind to a polypeptide present on the surface of a cancer cell. For example, a cell engager provided herein can include an antigen binding domain having the ability to bind to a NKG2C polypeptide, followed by an IL-15 polypeptide, followed by an antigen binding domain having the ability to bind to a polypeptide present on the surface of a cancer cell. For example, a cell engager provided herein can include an antigen binding domain having the ability to bind to a polypeptide present on the surface of a cancer cell, followed by an IL-15 polypeptide, followed by an antigen binding domain having the ability to bind to a NKG2C polypeptide.

In some cases, a cell engager provided herein can have a signal polypeptide located N-terminal to both a first antigen binding domain having the ability to bind to a NKG2C polypeptide and a second antigen binding domain having the ability to bind to a polypeptide present on the surface of a cancer cell (e.g., a CD33 polypeptide). For example, a cell engager provided herein can include a signal polypeptide, followed by an antigen binding domain having the ability to bind to a NKG2C polypeptide, followed by an antigen binding domain having the ability to bind to a polypeptide present on the surface of a cancer cell (e.g., a CD33 polypeptide).

When a cell engager provided herein includes one or more additional components (e.g., one or more additional polypeptides), the one or more additional components can be directly connected or can be connected using any appropriate linker sequence. In some cases, an antigen binding domain (e.g., an antigen binding domain having the ability to bind to a NKG2C polypeptide and/or an antigen binding domain having the ability to bind to a polypeptide present on the surface of a cancer cell) can be directly connected to an IL-15 polypeptide. In some cases, an antigen binding domain (e.g., an antigen binding domain having the ability to bind to a NKG2C polypeptide and/or an antigen binding domain having the ability to bind to a polypeptide present on the surface of a cancer cell) can be connected to an IL-15 polypeptide via a linker sequence. For example, a cell engager provided herein can include an antigen binding domain having the ability to bind to a NKG2C polypeptide, followed by a linker, followed by an IL-15 polypeptide, followed by a linker, followed by an antigen binding domain having the ability to bind to a polypeptide present on the surface of a cancer cell (e.g., a CD33 polypeptide). A linker can be any appropriate length. For example, a linker that can be used to connect an antigen binding domain (an antigen binding domain having the ability to bind to a NKG2C polypeptide and/or an antigen binding domain having the ability to bind to a polypeptide present on the surface of a cancer cell) and an additional polypeptide (e.g., an IL-15 polypeptide) can be from about 3 to about 100 (e.g., from about 3 to about 90, from about 3 to about 80, from about 3 to about 70, from about 3 to about 60, from about 3 to about 50, from about 3 to about 40, from about 3 to about 30, from about 3 to about 20, from about 3 to about 15, from about 5 to about 100, from about 10 to about 100, from about 20 to about 100, from about 30 to about 100, from about 40 to about 100, from about 50 to about 100, from about 60 to about 100, from about 70 to about 100, from about 10 to about 50, from about 10 to about 40, from about 10 to about 30, from about 10 to about 20, or from about 12 to about 17) amino acid residues in length. Examples of linker sequences that can be used to connect an IL-15 polypeptide to an antigen binding domain (e.g., an antigen binding domain having the ability to bind to a NKG2C polypeptide and/or an antigen binding domain having the ability to bind to a polypeptide present on the surface of a cancer cell) include, without limitation, GGGGSGGGGSGGGGS (SEQ ID NO:21), GGGGSGGGGSGGGGSGGGGS (SEQ ID NO:22), GGGGSGGGGSGGGGSGGGGSGGGGS (SEQ ID NO:23), GGGGSGGGGS (SEQ ID NO:63), GSTSGSGKPGSGEGSTKG (SEQ ID NO:41), PSGQAGAAASESLFVSNHAY (SEQ ID NO:64), EASGGPE (SEQ ID NO:65), EPKSSDKTHTSPPSPEL (SEQ ID NO:66), RATPSHNSHQVPSAGGPTANSGTSG (SEQ ID NO:67), and SSGGGGSGGGGGGSSRSSL (SEQ ID NO:68).

In some cases, a cell engager (e.g., a BiKE or a tri-specific killer engager) provided herein can be designed to target a NKG2C polypeptide and target a polypeptide expressed on the surface of a cancer cell (e.g., a CD33 polypeptide). For example, a cell engager provided herein can be designed to include an scFv having a heavy chain variable domain comprising SEQ ID NO:5, SEQ ID NO:6, and SEQ ID NO:7, followed by a linker, followed by a light chain variable domain comprising SEQ ID NO:8, SEQ ID NO:9, and SEQ ID NO:10, followed by a linker, followed by an scFv having a heavy chain variable domain comprising SEQ ID NO:24, SEQ ID NO:25, and SEQ ID NO:26, followed by a linker, followed by a light chain variable domain comprising SEQ ID NO:27, SEQ ID NO:28, and SEQ ID NO:29. For example, a cell engager provided herein can be designed to include an scFv having a heavy chain variable domain comprising SEQ ID NO:19, followed by a linker, followed by a light chain variable domain comprising SEQ ID NO:20, followed by a linker, followed by an scFv having a heavy chain variable domain comprising SEQ ID NO:38, followed by a linker, followed by a light chain variable domain comprising SEQ ID NO:39.

In some cases, a cell engager (e.g., a BiKE or a tri-specific killer engager) provided herein can be designed to target a NKG2C polypeptide and target a polypeptide expressed on the surface of a cancer cell (e.g., a CD33 polypeptide), and also to include an IL-15 polypeptide. For example, a cell engager provided herein can be designed to include an scFv having a heavy chain variable domain comprising SEQ ID NO:5, SEQ ID NO:6, and SEQ ID NO:7, followed by a linker, followed by a light chain variable domain comprising SEQ ID NO:8, SEQ ID NO:9, and SEQ ID NO:10, followed by a linker, followed by an IL-15 polypeptide (e.g., a human IL-15 polypeptide), followed by a linker, followed by an scFv having a heavy chain variable domain comprising SEQ ID NO:24, SEQ ID NO:25, and SEQ ID NO:26, followed by a linker, followed by a light chain variable domain comprising SEQ ID NO:27, SEQ ID NO:28, and SEQ ID NO:29. For example, a cell engager provided herein can be designed to include an scFv having a heavy chain variable domain comprising SEQ ID NO:19, followed by a linker, followed by a light chain variable domain comprising SEQ ID NO:20, followed by a linker, followed by an IL-15 polypeptide (e.g., a human IL-15 polypeptide), followed by a linker, followed by an scFv having a heavy chain variable domain comprising SEQ ID NO:38, followed by a linker, followed by a light chain variable domain comprising SEQ ID NO:39.

As indicated herein, the amino acid sequences described herein can include amino acid modifications (e.g., the articulated number of amino acid modifications). Such amino acid modifications can include, without limitation, amino acid substitutions, amino acid deletions, amino acid additions, and combinations. In some cases, an amino acid modification can be made to improve the binding and/or contact with an antigen and/or to improve a functional activity of a cell engager provided herein. In some cases, an amino acid substitution within an articulated sequence identifier can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution within an articulated sequence identifier can be a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

Methods for generating an amino acid sequence variant (e.g., an amino acid sequence that includes one or more modifications with respect to an articulated sequence identifier) can include site-specific mutagenesis or random mutagenesis (e.g., by PCR) of a nucleic acid encoding a cell engager or a portion thereof. See, for example, Zoller, Curr: Opin. Biotechnol. 3: 348-354 (1992). Both naturally occurring and non-naturally occurring amino acids (e.g., artificially-derivatized amino acids) can be used to generate an amino acid sequence variant provided herein.

A representative cell engager having the ability to bind to a NKG2C polypeptide (e.g., a human NKG2C polypeptide) and having the ability to bind to a CD33 polypeptide (e.g., a human CD33 polypeptide) is further described in Example 8.

The cell engagers provided herein can be produced using any appropriate method. For example, the cell engagers provided herein can be produced in recombinant host cells. For example, a nucleic acid encoding a cell engager provided herein can be constructed, introduced into an expression vector, and expressed in suitable host cells. Example 4, Example 7, and Example 9 are sequence listings of nucleic acid sequences encoding an exemplary cell engager described herein. In some cases, a cell engager provided herein can be recombinantly produced in prokaryotic hosts such as E. coli, Bacillus brevis, Bacillus subtilis, Bacillus megaterium, Lactobacillus zeae/casei, or Lactobacillus paracasei. A cell engager provided herein also can be recombinantly produced in eukaryotic hosts such as yeast (e.g., Pichia pastoris, Saccharomyces cerevisiae, Hansenula polymorpha, Schizosaccharomyces pombe, Schwanniomyces occidentalis, Kluyveromyces lactis, or Yarrowia lipolytica), filamentous fungi of the genera Trichoderma (e.g., T. reesei) and Aspergillus (e.g., A. niger and A. oryzae), protozoa such as Leishmania tarentolae, insect cells, or mammalian cells (e.g., mammalian cell lines such as Chinese hamster ovary (CHO) cells, Per.C6 cells, mouse myeloma NS0 cells, baby hamster kidney (BHK) cells, or human embryonic kidney cell line HEK293). See, for example, the Frenzel et al. reference (Front Immunol., 4:217 (2013)).

In some cases, a cell engager provided herein can be substantially pure. The term “substantially pure” as used herein with reference to a cell engager refers to the cell engager as being substantially free of other polypeptides, lipids, carbohydrates, and nucleic acid. Thus, a substantially pure cell engager provided herein is any cell engager that is at least 60 percent pure. A substantially pure cell engager provided herein can be at least about 65, 70, 75, 80, 85, 90, 95, or 99 percent pure.

In some cases, a cell engager provided herein can be fused or conjugated (e.g., covalently or non-covalently attached) to another polypeptide or other moiety to provide a fusion protein or conjugate. For example, a cell engager provided herein can be conjugated (e.g., covalently or non-covalently attached) to a polymer (e.g., polyethylene glycol (PEG), polyethylenimine (PEI) modified with PEG (PEI-PEG), and/or polyglutamic acid (PGA) (N-(2-Hydroxypropyl) methacrylamide (HPMA) copolymers), hyaluronic acid, a fluorescent substance, a luminescent substance, a hapten, an enzyme, a metal chelate, a drug, a radioisotope, and/or a cytotoxic agent. Any appropriate method can be used to conjugate (e.g., covalently or non-covalently attach) another polypeptide or other moiety to a cell engager provided herein. For example, another polypeptide or other moiety can be conjugated to a engager provided herein using the methods described in U.S. Pat. No. 8,021,661.

In some cases, a cell engager provided herein can be modified with a moiety that improves its stabilization and/or retention in circulation, for example, in blood, serum, or other tissues by, for example, at least 1.5-, 2-, 5-, 10-, or 50-fold. For example, a cell engager provided herein can be attached (e.g., covalently or non-covalently attached) to a polymer such as a substantially non-antigenic polymer. Examples of substantially non-antigenic polymers that can be used as described herein include, without limitation, polyalkylene oxides, and polyethylene oxides. In some cases, a polymer used herein can have any appropriate molecule weight. For example, a polymer having an average molecular weight from about 200 Daltons to about 35,000 Daltons (e.g., from about 1,000 to about 15,000 Daltons or from about 2,000 to about 12,500 Daltons) can be used. In some cases, a cell engager provided herein can be attached (e.g., covalently or non-covalently) to a water-soluble polymer. Examples of water-soluble polymers that can be used as described herein include, without limitation, hydrophilic polyvinyl polymers, polyvinylalcohol, polyvinylpyrrolidone, polyalkylene oxide homopolymers, polyethylene glycol (PEG), polypropylene glycols, and polyoxyethylenated polyols and copolymers thereof and/or block copolymers thereof provided that the water solubility of the copolymer or block copolymers is maintained.

In some cases, a cell engager provided herein can be attached (e.g., covalently or non-covalently attached) to one or more polyoxyalkylenes (e.g., polyoxyethylene, polyoxypropylene, or block copolymers of polyoxyethylene and polyoxypropylene), polymethacrylates, carbomers, branched or unbranched polysaccharides, or combinations thereof. For example, a cell engager provided herein can be covalently attached to polyoxyethylene.

This document also provides nucleic acid molecules (e.g., isolated nucleic acid molecules) having a nucleic acid sequence encoding at least part of a cell engager provided herein. For example, an isolated nucleic acid molecule provided herein can include a nucleic acid sequence encoding a heavy chain variable domain such as a heavy chain variable domain as set forth in Example 3 or Example 5. In some cases, an isolated nucleic acid molecule that can encode a heavy chain variable domain provided herein can be as set forth in Example 4 or Example 7. In another example, an isolated nucleic acid molecule provided herein can include a nucleic acid sequence encoding a light chain variable domain such as a light chain variable domain as set forth in Example 3 or Example 5. In some cases, an isolated nucleic acid molecule that can encode a light chain variable domain provided herein can be as set forth in Example 4 or Example 7. In some cases, an isolated nucleic acid molecule provided herein can include a nucleic acid sequence encoding both (a) a heavy chain variable domain and (b) a light chain variable domain, with or without, encoding a linker polypeptide. A nucleic acid provided herein (e.g., an isolated nucleic acid molecule) can be single stranded or double stranded nucleic acid of any appropriate type (e.g., DNA, RNA, or DNA/RNA hybrids).

This document also provides nucleic acid constructs containing one or more nucleic acids provided herein. In some cases, a nucleic acid construct containing one or more nucleic acids provided herein can be a vector (e.g., a plasmid vector or a viral vector). An example of a plasmid vector that can be designed to include one or more nucleic acids having a nucleic acid sequence encoding at least part of a cell engager provided herein includes, without limitation, phagemids. Examples of viral vectors that can be designed to include one or more nucleic acids having a nucleic acid sequence encoding at least part of a cell engager provided herein include, without limitation, retroviral vectors, parvovirus-based vectors (e.g., adenoviral-based vectors and adeno-associated virus (AAV)-based vectors), lentiviral vectors (e.g., herpes simplex (HSV)-based vectors), poxviral vectors (e.g., vaccinia virus-based vectors and fowlpox virus-based vectors), and hybrid or chimeric viral vectors. For example, a viral vector having an adenoviral backbone with lentiviral components such as those described elsewhere (Zheng et al., Nat. Biotech., 18(2): 176-80 (2000); WO 98/22143; WO 98/46778; and WO 00/17376) or viral vectors having an adenoviral backbone with AAV components such as those described elsewhere (Fisher et al., Hum. Gene Ther., 7:2079-2087 (1996)) can be designed to include one or more nucleic acids having a nucleic acid sequence encoding at least part of a cell engager provided herein.

In some cases, a nucleic acid construct (e.g., a vector such as a plasmid vector or a viral vector) provided herein can include a nucleic acid sequence encoding a full length cell engager provided herein. A nucleic acid construct (e.g., a vector such as a plasmid vector or a viral vector) provided herein can include any appropriate promoter and other regulatory sequence (e.g., transcription and translation initiation and termination codons) operably linked the nucleic acid sequence encoding at least part of a cell engager provided herein. In some cases, a promoter used to drive expression can be a constitutive promotor or a regulatable promotor. Examples of regulatable promoters that can be used as described herein include, without limitation, inducible promotors, repressible promotors, and tissue-specific promoters. Examples of viral promotors that can be used as described herein include, without limitation, adenoviral promotors, vaccinia virus promotors, CMV promotors (e.g., immediate early CMV promotors), and AAV promoters.

Any appropriate method can be used to make a nucleic acid construct (e.g., a vector such as a plasmid vector or a viral vector) provided herein having a nucleic acid sequence encoding at least part of a cell engager provided herein. For example, molecular cloning techniques can be used to make a nucleic acid construct (e.g., a vector such as a plasmid vector or a viral vector) provided herein having a nucleic acid sequence encoding at least part of a cell engager provided herein as described elsewhere (see, e.g., Sambrook et al., Molecular Cloning: A Laboratory Manual, 2nd edition, Cold Spring Harbor Laboratory, NY (1989); and Ausubel et al., Current Protocols in Molecular Biology, Green Publishing Associates and John Wiley & Sons, New York, N.Y. (1994)).

This document also provides host cells that include a nucleic acid provided herein (e.g., a nucleic acid having a nucleic acid sequence encoding at least part of a cell engager provided herein). Host cells that can be designed to include one or more nucleic acids provided herein can be prokaryotic cells or eukaryotic cells. Examples of prokaryotic cells that can be designed to include a nucleic acid provided herein include, without limitation, E. coli (e.g., Tb-1, TG-1, DH5a, XL-Blue MRF (Stratagene), SA2821, or Y1090 cells), Bacillus subtilis, Salmonella typhimurium, Serratia marcescens, or Pseudomonas (e.g., P. aerugenosa) cells. Examples of eukaryotic cells that can be designed to include a nucleic acid provided herein include, without limitation, insect cells (e.g., Sf9 or Ea4 cells), yeast cells (e.g., S. cerevisiae cells), and mammalian cells (e.g., mouse, rat, hamster, monkey, or human cells). For example, VERO cells, HeLa cells, 3T3 cells, chinese hamster ovary (CHO) cells, W138 BHK cells, COS-7 cells, and MDCK cells can be designed to include a nucleic acid provided herein. Any appropriate method can be used to introduce one or more nucleic acids provided herein (e.g., a vector such as a plasmid vector or viral vector having a nucleic acid sequence encoding at least part of a cell engager provided herein) into a host cell. For example, calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, electroporation, or combinations thereof can be used to introduce a nucleic acid provided herein into a host cell (see, e.g., Sambrook et al., Molecular Biology: A Laboratory Manual, Cold Spring Harbor Laboratory, NY (1989); Davis et al., Basic Methods in Molecular Biology (1986); and Neumann et al., EMBO J., 1:841 (1982)).

In some cases, a cell engager provided herein can be produced using a method that includes (a) introducing nucleic acid encoding the polypeptide into a host cell; (b) culturing the host cell in culture medium under conditions sufficient to express the polypeptide; (c) harvesting the polypeptide from the cell or culture medium; and (d) purifying the polypeptide (e.g., to reach at least 50, 60, 70, 80, 90, 95, 97, 98, or 99 percent purity).

In some cases, a cell engager provided herein, a nucleic acid provided herein (e.g., nucleic acid encoding a full length cell engager provided herein), a vector provided herein (e.g., a viral vector designed to express a full length cell engager provided herein), and/or a host cell provided herein (e.g., a host cell designed to express a full length cell engager provided herein) can be formulated as a pharmaceutical composition for administration to a mammal (e.g. a human) having cancer (e.g., a leukemia such as AML) to treat that mammal. In some cases, a cell engager provided herein, a nucleic acid provided herein (e.g., nucleic acid encoding a full length cell engager provided herein), a vector provided herein (e.g., a viral vector designed to express a full length cell engager provided herein), and/or a host cell provided herein (e.g., a host cell designed to express a full length cell engager provided herein) can be formulated as a pharmaceutical composition for administration to a mammal (e.g. a human) to reduce the number of cancer cells within the mammal and/or to increase the survival of the mammal suffering from cancer. For example, a cell engager provided herein having the ability to bind to a NKG2C polypeptide (e.g., a human NKG2C polypeptide) and having the ability to bind to a polypeptide present on the surface of a cancer cell (e.g., a CD33 polypeptide) can be formulated as a pharmaceutical composition for administration to a mammal (e.g. a human). In some cases, a pharmaceutical composition provided herein can include a pharmaceutically acceptable carrier such as a buffer, a salt, a surfactant, a sugar, a tonicity modifier, or combinations thereof as, for example, described elsewhere (Gervasi, et al., Eur. J. Pharmaceutics and Biopharmaceutics, 131:8-24 (2018)). Examples of pharmaceutically acceptable carriers that can be used to make a pharmaceutical composition provided herein include, without limitation, water, lactic acid, citric acid, sodium chloride, sodium citrate, sodium succinate, sodium phosphate, a surfactant (e.g., polysorbate 20, polysorbate 80, or poloxamer 188), dextran 40, or a sugar (e.g., sorbitol, mannitol, sucrose, dextrose, or trehalose), or combinations thereof. For example, a pharmaceutical composition designed to include a cell engager provided herein (or a nucleic acid, a vector, or a host cell provided herein) can be formulated to include a buffer (e.g., an acetate, citrate, histidine, succinate, phosphate, or hydroxymethylaminomethane (Tris) buffer), a surfactant (e.g., polysorbate 20, polysorbate 80, or poloxamer 188), and a sugar such as sucrose. Other ingredients that can be included within a pharmaceutical composition provided herein include, without limitation, amino acids such as glycine or arginine, antioxidants such as ascorbic acid, methionine, or ethylenediaminetetraacetic acid (EDTA), anticancer agents such as enzalutamide, imanitib, gefitinib, erlotini, sunitinib, lapatinib, nilotinib, sorafenib, temsirolimus, everolimus, pazopanib, crizotinib, ruxolitinib, axitinib, bosutinib, cabozantinib, ponatinib, regorafenib, ibrutinib, trametinib, perifosine, bortezomib, carfilzomib, batimastat, ganetespib, obatoclax, navitoclax, taxol, paclitaxel, or bevacizumab, or combinations thereof. For example, a pharmaceutical composition provided herein can be formulated to include one or more cell engagers provided herein in combination with one or more checkpoint inhibitors such as anti-PD-1 antibodies or PD-1 inhibitors (e.g., cemiplimab, nivolumab, pembrolizumab, JTX-4014, spartalizumab, camrelizumab, sintilimab, tislelizumab, toripalimab, dostarlimab, INCMGA00012, AMP-224, or AMP-514), anti-PD-L1 antibodies or PD-L1 inhibitors (e.g., avelumab, durvalumab, atezolizumab, KN035, CK-301, AUNP12, CA-170, or BMS-986189), and/or anti-CTLA-4 antibodies (e.g., ipilimumab).

In some cases, when a pharmaceutical composition is formulated to include one or more cell engagers provided herein, any appropriate concentration of the cell engagers can be used. For example, a pharmaceutical composition provided herein can be formulated to be a liquid that includes from about 1 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a cell engager provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a cell engager provided herein. In some cases, when a pharmaceutical composition is formulated to include one or more nucleic acids (e.g., vectors such as viral vectors) encoding at least part of a cell engager provided herein (e.g., a full length cell engager provided herein), any appropriate concentration of the nucleic acid can be used. For example, a pharmaceutical composition provided herein can be formulated to be a liquid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a nucleic acid provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a nucleic acid provided herein.

In some cases, a pharmaceutical composition designed to include a cell engager provided herein can be formulated to include one or more agents capable of reducing aggregation of the cell engager when formulated. Examples of such agents that can be used as described herein include, without limitation, methionine, arginine, lysine, aspartic acid, glycine, glutamic acid, and combinations thereof. In some cases, one or more of these amino acids can be included within the formulation at a concentration from about 0.5 mM to about 145 mM (e.g., from about 1 mM to about 145 mM, from about 10 mM to about 145 mM, from about 100 mM to about 145 mM, from about 0.5 mM to about 125 mM, from about 0.5 mM to about 100 mM, from about 0.5 mM to about 75 mM, or from about 10 mM to about 100 mM).

A pharmaceutical composition provided herein can be in any appropriate form. For example, a pharmaceutical composition provided herein can designed to be a liquid, a semi-solid, or a solid. In some cases, a pharmaceutical composition provided herein can be a liquid solution (e.g., an injectable and/or infusible solution), a dispersion, a suspension, a tablet, a pill, a powder, a microemulsion, a liposome, or a suppository. In some cases, a pharmaceutical composition provided herein can be lyophilized. In some cases, a pharmaceutical composition provided herein (e.g., a pharmaceutical composition that includes one or more cell engagers provided herein can be formulated with a carrier or coating designed to protect against rapid release. For example, a pharmaceutical composition provided herein can be formulated as a controlled release formulation or as a regulated release formulation as described elsewhere (U.S. Patent Application Publication Nos. 2019/0241667; 2019/0233522; and 2019/0233498).

This document also provides methods for administering a composition (e.g., a pharmaceutical composition provided herein) containing one or more cell engagers provided herein (or a nucleic acid, vector, or host cell provided herein) to a mammal (e.g., a human). For example, a composition (e.g., a pharmaceutical composition provided herein) containing one or more cell engagers provided herein (or a nucleic acid, vector, and/or host cell provided herein) can be administered to a mammal (e.g., a human) having cancer (e.g., a leukemia such as AML) to treat that mammal. In some cases, a composition (e.g., a pharmaceutical composition provided herein) containing one or more cell engagers provided herein (or a nucleic acid, vector, and/or host cell provided herein) can be administered to a mammal (e.g. a human) to reduce the number of cancer cells within the mammal and/or to increase the survival of the mammal suffering from cancer.

In some cases, a composition (e.g., a pharmaceutical composition provided herein) containing one or more cell engagers provided herein (or a nucleic acid, vector, and/or host cell provided herein) can be administered to a mammal (e.g. a human) having cancer (e.g., a leukemia such as AML) to reduce or eliminate one or more symptoms of the cancer. Examples of symptoms of a cancer (e.g., a leukemia such as AML) that can be reduced using a composition comprising one or more cell engagers described herein include, without limitation, fever, bone pain, lethargy and fatigue, shortness of breath, pale skin, frequent infections, easy bruising, unusual bleeding (e.g., frequent nosebleeds and bleeding from the gums), and neutropenia.

Any appropriate cancer can be treated using a composition (e.g., a pharmaceutical composition provided herein) containing one or more cell engagers provided herein (or a nucleic acid, vector, or host cell provided herein). For example, a mammal (e.g., a human) having cancer can be treated by administering a composition (e.g., a pharmaceutical composition) containing one or more cell engagers provided herein to that mammal. In some cases, a cancer that can be treated as described herein can include one or more solid tumors. In some cases, a cancer that can be treated as described herein can be a blood cancer. Examples of cancers that can be treated as described herein include, without limitation, leukemias (e.g., AML), lymphomas, myelodysplastic syndromes (MDS), and systemic mastocytosis. In some cases, a mammal (e.g., a human) having cancer (e.g., a leukemia such as AML) can be administered a composition (e.g., a pharmaceutical composition) containing one or more cell engagers provided herein to treat that mammal (e.g., to reduce the number of cancer cells within the mammal).

Any appropriate method can be used to administer a composition (e.g., a pharmaceutical composition) provided herein to a mammal (e.g., a human). For example, a composition provided herein (e.g., a pharmaceutical composition containing one or more cell engagers provided herein) can be administered to a mammal (e.g., a human) intravenously (e.g., via an intravenous injection or infusion), subcutaneously (e.g., via a subcutaneous injection), intraperitoneally (e.g., via an intraperitoneal injection), orally, via inhalation, or intramuscularly (e.g., via intramuscular injection). In some cases, the route and/or mode of administration of a composition (e.g., a pharmaceutical composition provided herein) can be adjusted for the mammal being treated.

In some cases, an effective amount of a composition containing cell engagers provided herein (or a nucleic acid, vector, or host cell provided herein) (e.g., a pharmaceutical composition provided herein) can be an amount that reduces the number of cancer cells within a mammal having cancer (e.g., a leukemia such as AML) without producing significant toxicity to the mammal. In some cases, an effective amount of a composition containing one or more cell engagers provided herein (or a nucleic acid, vector, or host cell provided herein) (e.g., a pharmaceutical composition provided herein) can be an amount that increases the survival time of a mammal having cancer (e.g., a leukemia such as AML) as compared to a control mammal having comparable cancer and not treated with the composition. For example, an effective amount of a cell engager provided herein can be from about 0.001 mg/kg to about 100 mg/kg (e.g., from about 0.001 mg/kg to about 90 mg/kg, from about 0.001 mg/kg to about 80 mg/kg, from about 0.001 mg/kg to about 70 mg/kg, from about 0.001 mg/kg to about 60 mg/kg, from about 0.001 mg/kg to about 50 mg/kg, from about 0.001 mg/kg to about 40 mg/kg, from about 0.001 mg/kg to about 30 mg/kg, from about 0.005 mg/kg to about 100 mg/kg, from about 0.01 mg/kg to about 100 mg/kg, from about 0.05 mg/kg to about 100 mg/kg, from about 0.1 mg/kg to about 100 mg/kg, from about 0.5 mg/kg to about 100 mg/kg, from about 1 mg/kg to about 100 mg/kg, from about 5 mg/kg to about 100 mg/kg, from about 0.01 mg/kg to about 25 mg/kg, from about 0.1 mg/kg to about 30 mg/kg, from about 0.15 mg/kg to about 25 mg/kg, from about 0.2 mg/kg to about 20 mg/kg, from about 0.5 mg/kg to about 20 mg/kg, from about 1 mg/kg to about 30 mg/kg, from about 1 mg/kg to about 25 mg/kg, from about 1 mg/kg to about 20 mg/kg, from about 2 mg/kg to about 20 mg/kg, from about 5 mg/kg to about 30 mg/kg, from about 10 mg/kg to about 30 mg/kg, from about 15 mg/kg to about 30 mg/kg, from about 20 mg/kg to about 30 mg/kg, from about 3 mg/kg to about 30 mg/kg, from about 0.5 mg/kg to about 10 mg/kg, from about 1 mg/kg to about 10 mg/kg, from about 1 mg/kg to about 5 mg/kg, or from about 1 mg/kg to about 3 mg/kg). The effective amount can remain constant or can be adjusted as a sliding scale or variable dose depending on the mammal's response to treatment. Various factors can influence the actual effective amount used for a particular application. For example, the severity of cancer when treating a mammal having cancer (e.g., a leukemia such as AML), the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective amount of a composition provided herein (e.g., a pharmaceutical composition containing one or more cell engagers provided herein) that is administered.

In some cases, an effective frequency of administration of a composition containing one or more cell engagers provided herein (or a nucleic acid, vector, or host cell provided herein) (e.g., a pharmaceutical composition provided herein) can be a frequency that reduces the number of cancer cells within a mammal having cancer (e.g., a leukemia such as AML) without producing significant toxicity to the mammal. In some cases, an effective frequency of administration of a composition containing one or more cell engagers provided herein (or a nucleic acid, vector, or host cell provided herein) (e.g., a pharmaceutical composition provided herein) can be a frequency that increases the survival time of a mammal having cancer (e.g., a leukemia such as AML) as compared to a control mammal having comparable cancer and not treated with the composition. For example, an effective frequency of administration of a pharmaceutical composition provided herein such as a pharmaceutical composition containing one or more cell engagers provided herein can be from about twice daily to about once a year (e.g., from about twice daily to about once a month, from about twice daily to about once a week, from about once daily to about once a month, or from one once daily to about once a week). In some cases, the frequency of administration of a pharmaceutical composition provided herein such as a pharmaceutical composition containing one or more cell engagers provided herein can be daily. The frequency of administration of a pharmaceutical composition provided herein such as a pharmaceutical composition containing one or more cell engagers provided herein can remain constant or can be variable during the duration of treatment. Various factors can influence the actual effective frequency used for a particular application. For example, the severity of the cancer (e.g., a leukemia such as AML), the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective frequency of administration of a composition provided herein (e.g., a pharmaceutical composition containing one or more cell engagers provided herein).

In some cases, an effective duration of administration of a composition containing one or more cell engagers provided herein (or a nucleic acid, vector, or host cell provided herein) (e.g., a pharmaceutical composition provided herein) can be a duration that reduces the number of cancer cells within a mammal without producing significant toxicity to the mammal. In some cases, an effective duration of administration of a composition containing one or more cell engagers provided herein (or a nucleic acid, vector, or host cell provided herein) (e.g., a pharmaceutical composition provided herein) can be a duration that increases the survival time of a mammal having cancer (e.g., a leukemia such as AML) as compared to a control mammal having comparable cancer and not treated with the composition. For example, an effective duration of administration of a pharmaceutical composition provided herein such as a pharmaceutical composition containing one or more cell engagers provided herein can vary from a single time point of administration to several weeks to several months (e.g., 4 to 12 weeks). Multiple factors can influence the actual effective duration used for a particular application. For example, the severity of the cancer (e.g., a leukemia such as AML), the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective duration of administration of a composition provided herein (e.g., a pharmaceutical composition containing one or more cell engagers provided herein).

In some cases, a composition (e.g., a pharmaceutical composition provided herein) containing one or more cell engagers provided herein (or a nucleic acid, vector, and/or host cell provided herein) can be administered to a mammal (e.g., a human) having cancer (e.g., a leukemia such as AML) together with a population of NK cells (e.g., in an adoptive cell therapy). Any appropriate NK cells can be administered to a mammal (e.g., a human) having cancer (e.g., a leukemia such as AML) together with one or more one or more cell engagers provided herein (or a nucleic acid, vector, and/or host cell provided herein). In some cases, at least some of the NK cells in a population of NK cells administered to a mammal can be NKG2C⁺ NK cells (e.g., can be engineered to be NKG2C⁺ NK cells). In some cases, at least some of the NK cells in a population of NK cells administered to a mammal can be iNK cells. In some cases, at least some of the NK cells in a population of NK cells administered to a mammal can be engineered to express one or more signaling polypeptides (e.g., a DAP12 polypeptide).

A population of NK cells can include any appropriate number of NK cells. For example, an effective amount of NK cells (e.g., NKG2C⁺ NK cells) that can be administered to a mammal (e.g., a human) having cancer (e.g., a leukemia such as AML) together with one or more one or more cell engagers provided herein (or a nucleic acid, vector, and/or host cell provided herein) can be from about 100 million NK cells per killigram (kg) body weight of the mammal (cells/kg) to about 900 million NK cells/kg (e.g., from about 100 million to about 800 million NK cells/kg, from about 100 million to about 700 million NK cells/kg, from about 100 million to about 600 million NK cells/kg, from about 100 million to about 500 million NK cells/kg, from about 100 million to about 400 million NK cells/kg, from about 100 million to about 300 million NK cells/kg, from about 100 million to about 200 million NK cells/kg, from about 200 million to about 900 million NK cells/kg, from about 300 million to about 900 million NK cells/kg, from about 400 million to about 900 million NK cells/kg, from about 500 million to about 900 million NK cells/kg, from about 600 million to about 900 million NK cells/kg, from about 700 million to about 900 million NK cells/kg, from about 800 million to about 900 million NK cells/kg, from about 200 million to about 800 million NK cells/kg, from about 300 million to about 700 million NK cells/kg, from about 400 million to about 600 million NK cells/kg, from about 200 million to about 400 million NK cells/kg, from about 300 million to about 500 million NK cells/kg, from about 400 million to about 600 million NK cells/kg, from about 500 million to about 700 million NK cells/kg, or from about 600 million to about 800 million NK cells/kg).

The invention will be further described in the following examples, which do not limit the scope of the invention described in the claims.

### EXAMPLES

### Example 1: Anti-NKG2C/IL-15/Anti-CD33 Killer Engager Directs Primary and iPSC-Derived NKG2C⁺ NK Cells to Specifically Target Myeloid Leukemia

This Example describes the design and characterization of molecules that can bind to a NKG2C polypeptide. An anti-NKG2C/IL-15/anti-CD33 killer engager (NKG2C-KE) was designed that can direct NKG2C⁺ cells to target CD33⁺ cells, a tumor associated antigen expressed by AML cells, to elicit a tumor-directed response from NKG2C⁺ NK cells.

### Results

### Functional Validation of a Novel NKG2C-KE

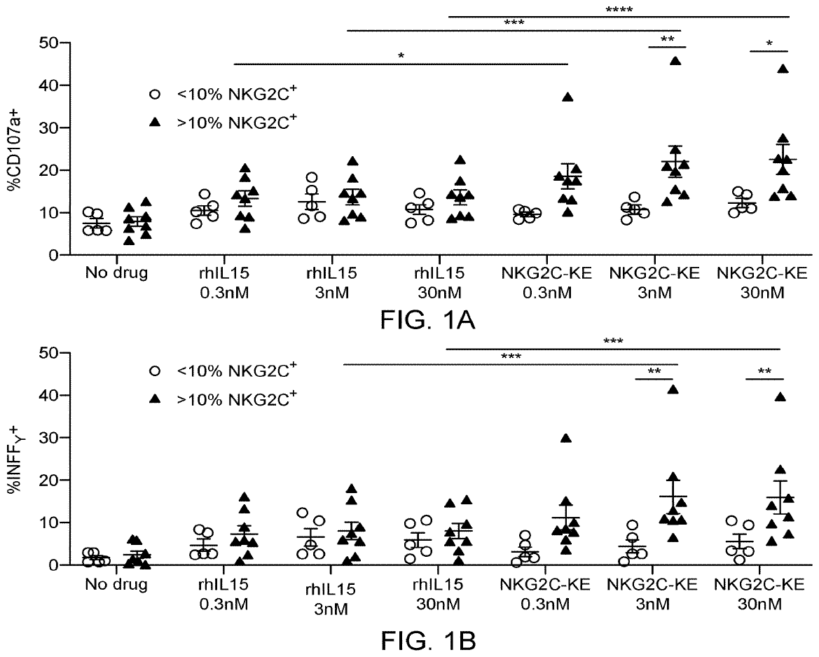

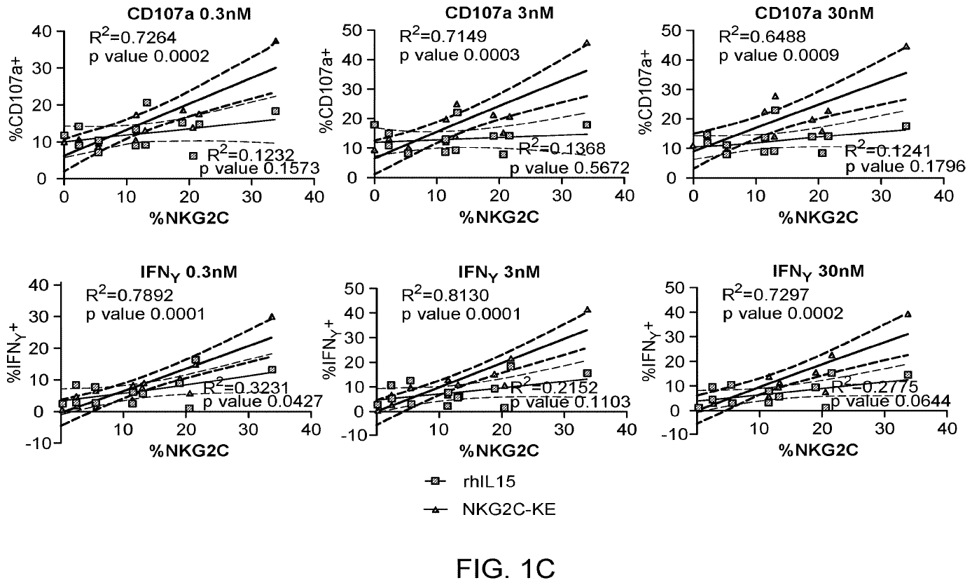

To determine if NKG2C engagement can be leveraged to specifically activate NK cells, a NKG2C-KE was generated using the sequence encoding an anti-NKG2C antibody (Cichocki et al., Sci. Transl. Med. 12:eaaz5618 (2020)). The variable heavy and light chain sequences of the anti-NKG2C antibody were cloned into a construct containing sequences encoding an anti-CD33 short-chain variable fragment (scFv) component to target AML and a wild-type IL-15 component sequence to link the two scFvs. Peripheral blood from healthy CMV seropositive donors was screened by flow cytometry for the presence of NKG2C⁺ NK cells and then placed into two groups based on whether greater than (NKG2C^{high}) or less than (NKG2C^{low}) 10% of NK cells were NKG2C⁺. Peripheral blood mononuclear cells (PBMCs) were isolated from each donor and co-cultured with the CD33⁺ AML cell line THP-1 with no treatment, rhIL-15, or the NKG2C-KE in a 5-hour flow cytometric functional assay. When compared to NKG2C^{high} cells cultured with rhIL-15, NKG2C^{high} cells cultured with NKG2C-KE exhibited increased degranulation at 0.3 nM (18.5% vs 13.3%, p=0.05), 3 nM (21.9% vs 13.9%, p=0.0001), and 30 nM concentrations (22.6% vs 13.6%, p<0.0001) (FIG. 1A). When comparing NKG2C^{low} and NKG2C^{high} NK cells, the NKG2C-KE triggered more robust degranulation for NKG2C^{high} NK cells at the 3 nM (10.7% vs 21.9%, p=0.009) and 30 nM (12.2% vs 22.6%, p=0.02) concentrations (FIG. 1A). Similar to the degranulation response, NKG2C^{high} NK cells produced more IFNγ in response to the NKG2C-KE compared to rhIL-15. Compared to rhIL-15, IFNγ was increased with the NKG2C-KE at 3 nM (8.1% vs 16.1%, p=0.0001) and 30 nM concentrations (8.0% vs 15.9%, p=0.0002). When comparing NKG2C^{low} to NKG2C^{high} frequency donors, the NKG2C^{high} NK cells produced more IFNγ with the NKG2C-KE at 3 nM (4.4% vs 16.1%, p=0.01) and 30 nM (5.5% vs 15.9%, p=0.04). There were no statistically significant differences in degranulation or IFNγ production were observed for NKG2C^{low} NK cells treated with NKG2C-KE compared to rhIL-15 (FIG. 1B). To evaluate whether NK cell function correlated with the frequency of NKG2C expression, a linear regression analysis was performed. While there was no association between degranulation and the frequency of NKG2C expression on NK cells in rhIL-15 cultures, NK cells stimulated with the NKG2C-KE exhibited higher frequencies of degranulation that correlated with the percentages of NKG2C expression at all concentrations (0.3 nM (R²=0.7, p=0.0002), 3 nM (R 2=0.7, p=0.0003) and 30 nM (R²=0.6, p=0.0009) (FIG. 1C). Frequencies of IFNγ production were also associated with NKG2C frequencies when NK cells were stimulated with various concentrations of the NKG2C-KE. Taken together, these data indicated that the NKG2C-KE specifically activates NK cells based on NKG2C expression.

The NKG2C-KE Activates and Expands NKG2C+ NK Cells from HCT Patients that Experienced CMV Reactivation

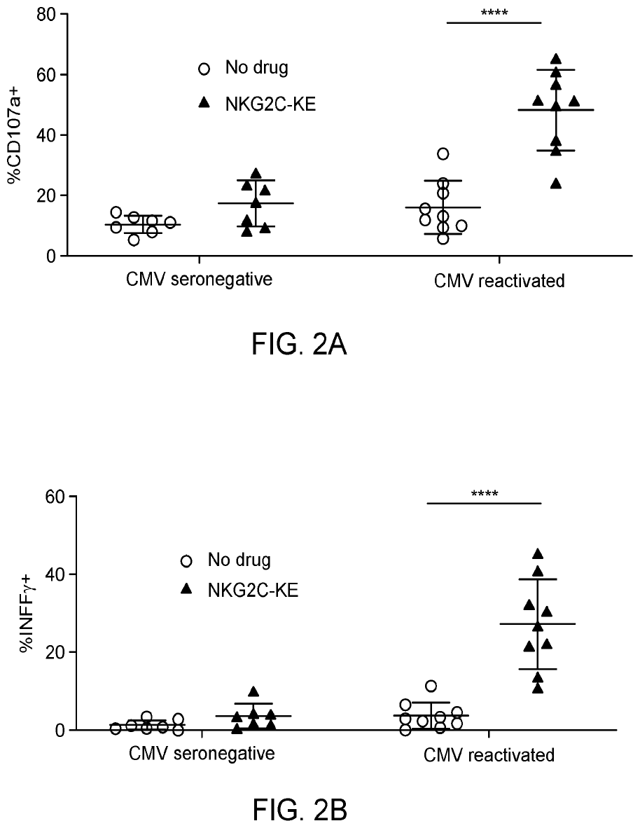

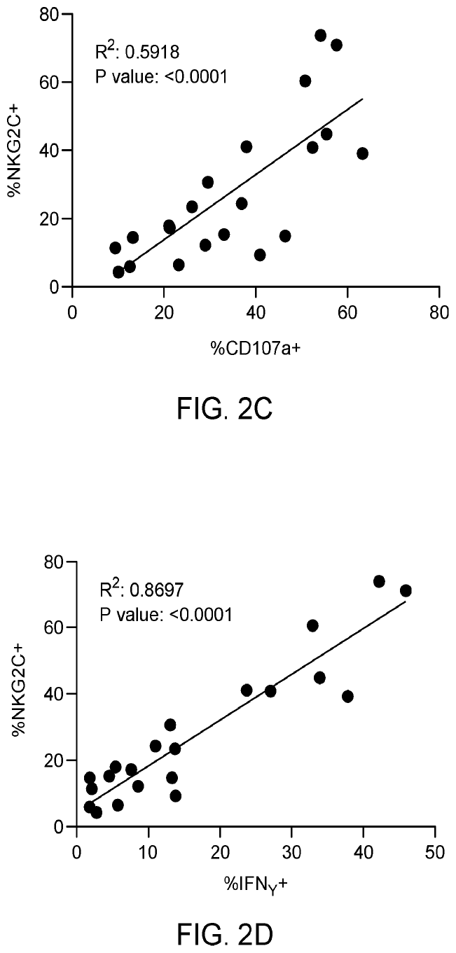

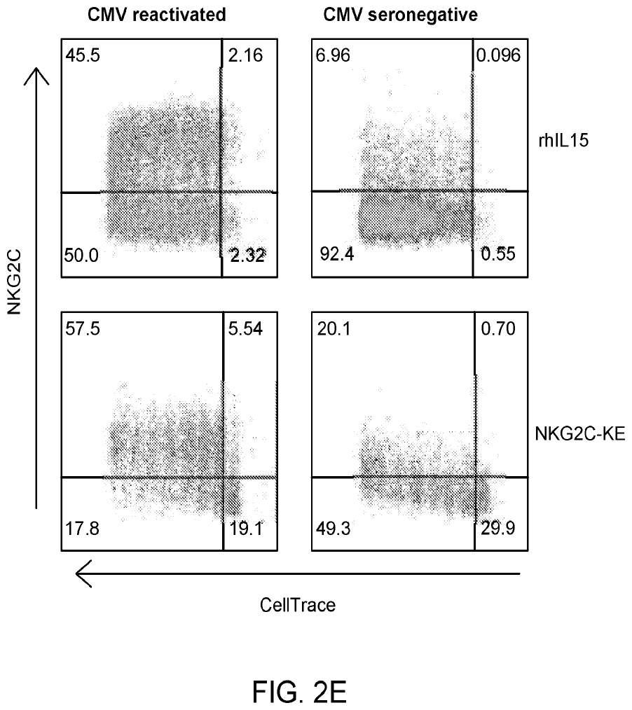

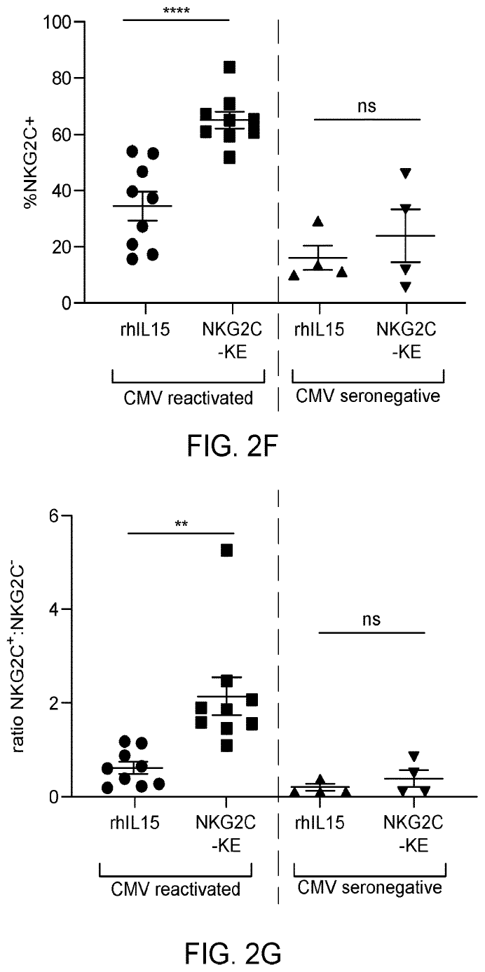

Reconstitution of an ‘adaptive’ NK cell (defined as CD57⁺NKG2C⁺) subset was found predominantly in transplant recipients who were CMV seropositive and experienced CMV reactivation post-transplant. This association suggested enhanced anti-tumor function by adaptive NKG2C⁺ NK cells compared to canonical NK cells lacking NKG2C expression. Thus, it was evaluated whether NKG2C-KE would preferentially activate NK cell function in transplant recipients who reactivated CMV compared to patients who were CMV seronegative. PBMCs collected from patients 6 months post-transplant were co-cultured with THP-1 AML cells, which express high levels of CD33, in the presence or absence of the NKG2C-KE. Compared to the control no treatment condition, CMV-reactivated patient NK cells degranulated more than 2-fold greater in the presence of the NKG2C-KE (16% vs 48.2%, p<0.0001) (FIG. 2A) and produced significantly more IFNγ (4.3% vs 29.8%, p=<0.0001) (FIG. 2B). However, this effect was not observed when testing NK cells from post-transplant CMV seronegative patients who reconstitute with low frequencies of NKG2C⁺ NK cells (FIGS. 2A and 2B).

Due to the differences in frequencies and quality of NKG2C⁺ NK cells between CMV seronegative patients and patients that reactivated CMV, it was sought to determine whether the responsiveness to the NKG2C-KE correlated with the frequencies of NKG2C. A high correlation between NKG2C expression and degranulation (R²=0.6, p<0.0001) as well as IFNγ production (R²=0.9, p<0.0001) were found further confirming the specificity of the NKG2C-KE (FIGS. 2C and 2D). Post-transplant PBMCs were then used to test whether the NKG2C-KE could induce selective proliferation of adaptive NK cells. Specific proliferation of NKG2C⁺ NK cells from CMV-reactivated patients was observed with the NKG2C-KE when compared to rhIL-15, suggesting a targeted delivery of the IL-15 by NKG2C-KE (FIG. 2E). While rhIL-15 resulted in broad proliferation of all NK cells, the IL-15 in the context of the NKG2C-KE was selective to the NKG2C⁺ population. When evaluating the proportion of NK cells that proliferate in response to the NKG2C-KE, NKG2C⁺ cells constituted a larger proportion of proliferating cells than those treated with rhIL-15 (FIG. 2F: 65.2% vs. 34.8%, p<0.0001) resulting in a higher NKG2C⁺:NKG2C⁻ ratio with the NKG2C-KE (FIG. 2G). In samples where NK cells contained no, or low frequencies of NKG2C, rhIL-15 and the NKG2C-KE triggered similar proportions of NK cell proliferation.

NKG2C-KE Controls CD33⁺ AML and Results in Enhanced Persistence of NKG2C⁺ Adaptive NK Cells In Vivo

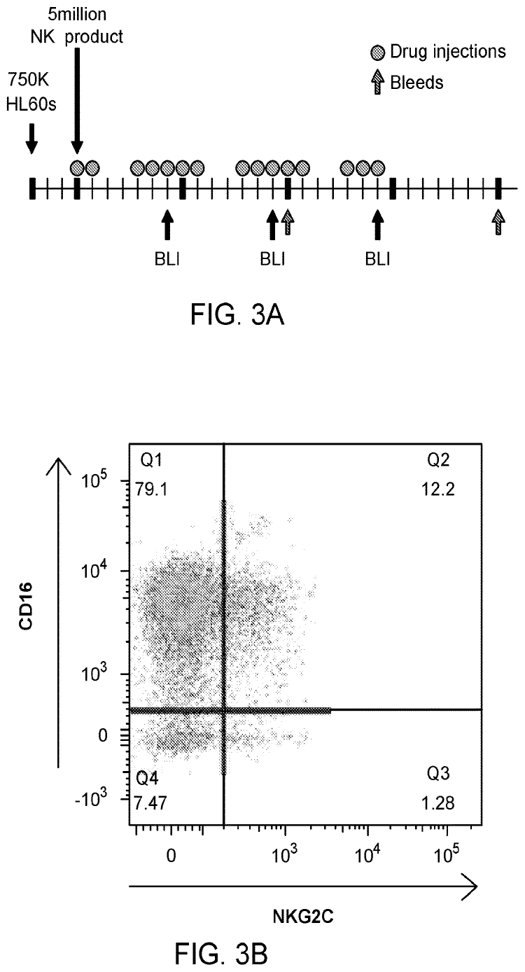

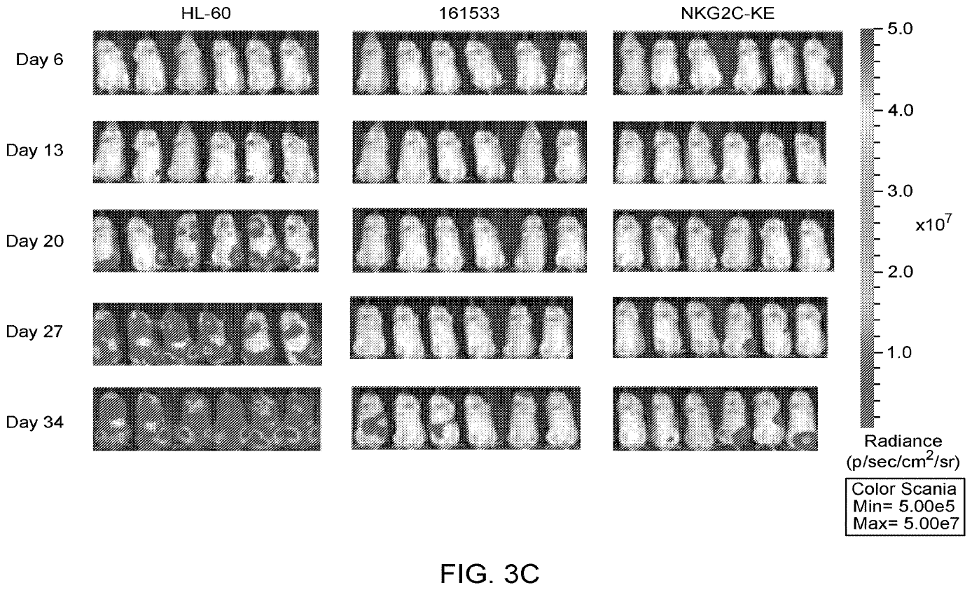

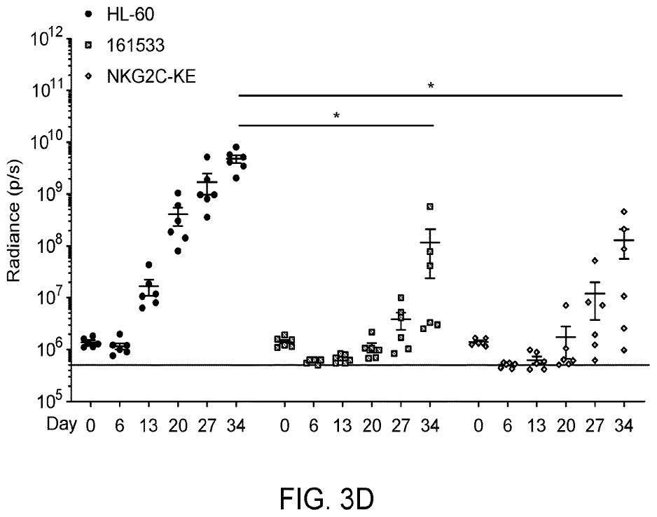

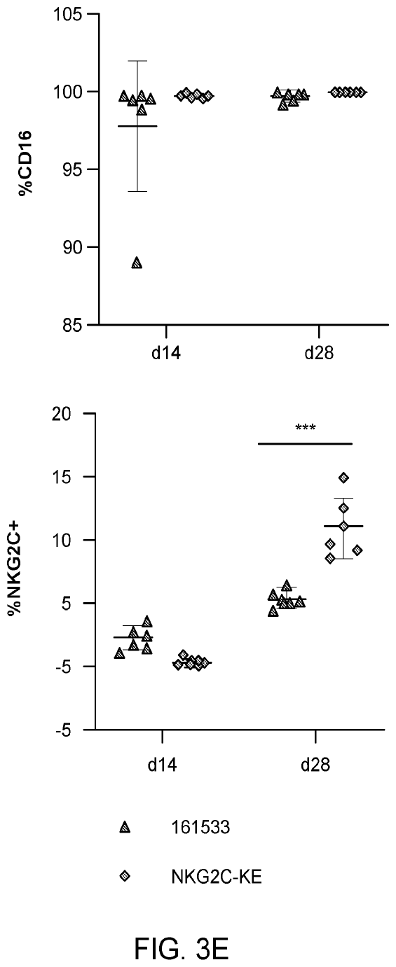

Based on the in vitro data showing specificity of NKG2C⁺ NK cells and the NKG2C-KE, findings were validated in vivo. A myeloid leukemia model was used where NOD scid gamma (NSG) mice that lack mouse lymphocytes (T, B and NK cells) were injected with a luciferase labelled CD33⁺ HL-60 myeloid tumor. AML was established, and three days later animals were treated with expanded PB NK cells from a CMV positive normal donor comprised of 13.5% NKG2C⁺ and 91.3% CD16′ NK cells (FIGS. 3A and 3B). NKG2C-KE or a CD161533 TriKE were administered 5 times weekly for 3 weeks with bioluminescence imaging (BLI) performed weekly. Blood was sampled on days 14 and day 28 to evaluate for NK cell persistence and expansion. In comparison to control animals (average radiance: 4.8e9+/−2.1e9), both the 161533 TriKE or NKG2C-KE in combination with NK cells mediated potent tumor control (1.2e8+/−2.3e8 vs 1.3e8+/−1.8e8, p=0.99) (FIGS. 3C and 3D), despite the 7-fold difference in CD16 vs NKG2C target for the immune engager on expanded PB NK cells (FIG. 3B). 28 days after adoptive transfer NK cells showed greater persistence of NKG2C⁺ NK cells when stimulated with the NKG2C-KE treated compared to 161533 TriKE, showing preferential persistence/expansion of adaptive NK cells in vivo. At day 14, the decreased NKG2C on NK cells with NKG2C-KE treatment was a result of receptor occupancy as the mice undergoing active NKG2C-KE treatment blocked NKG2C detection by flow cytometry (FIG. 3E).

Generation and Functional Attributes of iPSC-Derived NK Cells with Transgenic Expression of NKG2C

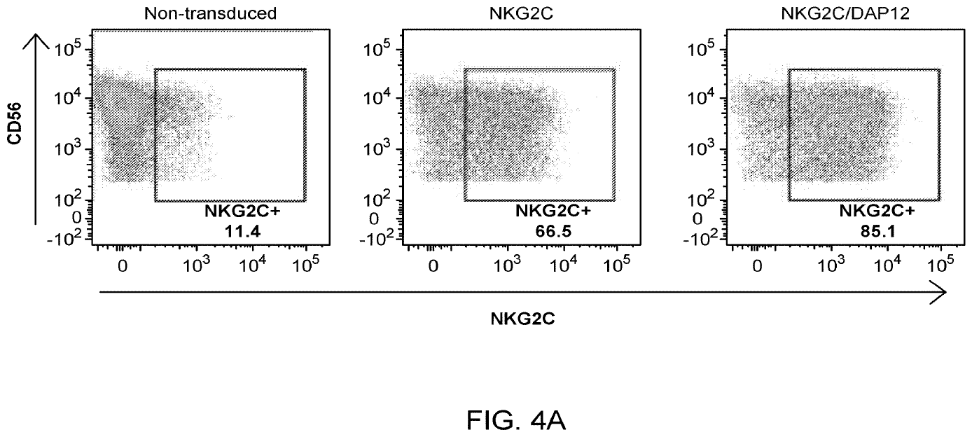

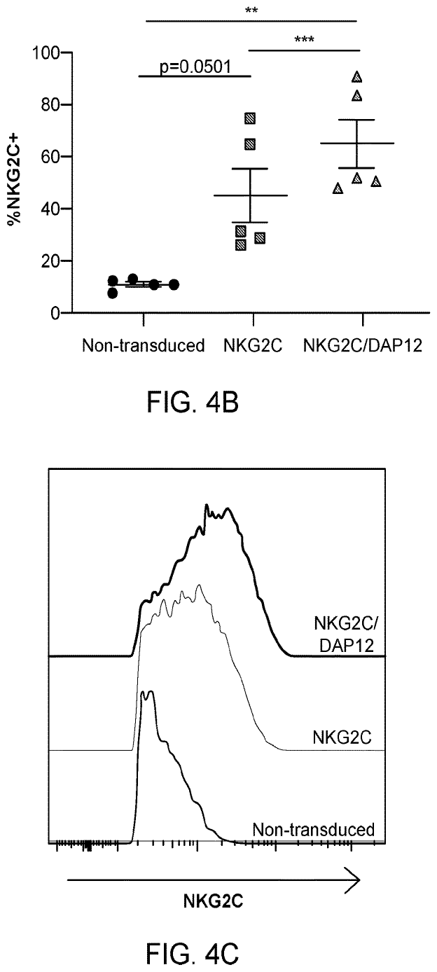

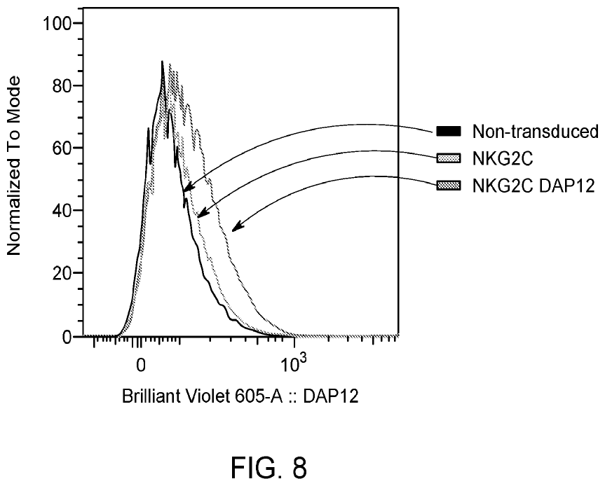

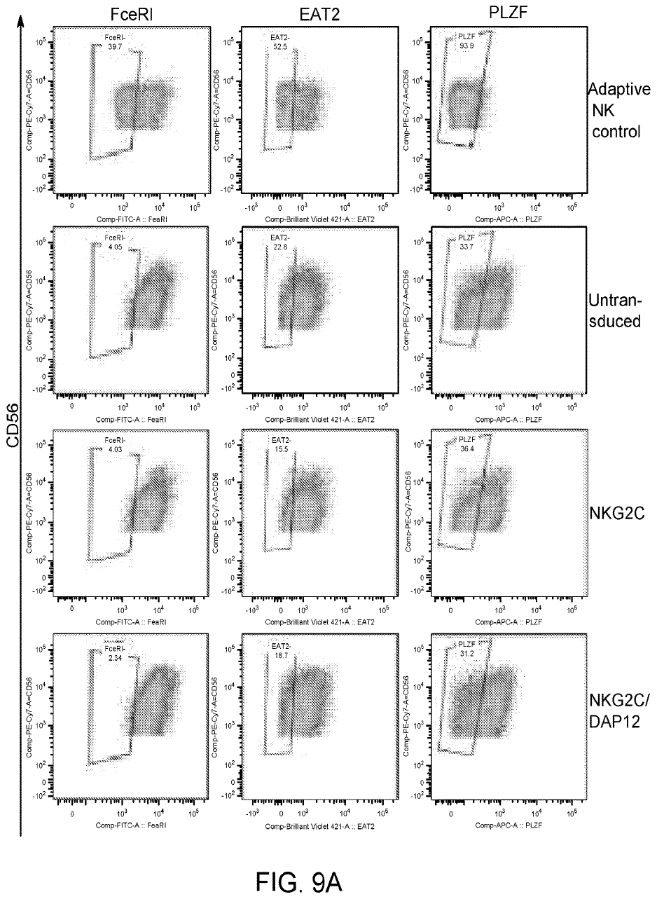

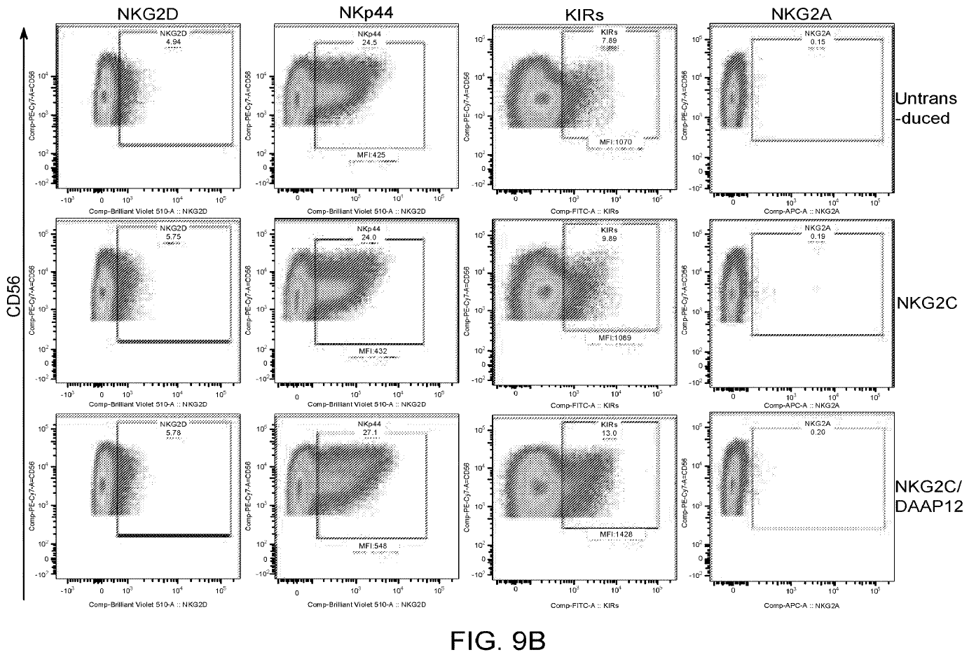

There is high variability in the frequency of NKG2C-expressing adaptive NK cells in the general population, and a minority of individuals harbor frequencies of these cells greater than 10% in their peripheral blood. To broaden the application of the NKG2C-KE, iPSC-derived NK cells were used to develop an ideal off-the shelf NK cell to combine with NKG2C-KE. Using this platform, two different iNK cell lines were created. One iNK cell line was engineered with NKG2C alone, and the other with both NKG2C and its signaling adaptor molecule, DAP12, for enhanced NKG2C expression and response. iPSCs were transduced, enriched for NKG2C expression, and banked to create a renewable starting material. Cells were then differentiated in a stepwise fashion through the CD34⁺ stage. CD34⁺ cells were then differentiated along the NK cell lineage. After expansion, the non-transduced parental iNK expressed low levels of NKG2C. NKG2C-transduced iNK cells displayed significantly higher surface expression of NKG2C, which was further increased in iNK cells expressing both NKG2C and DAP12 (FIG. 4A). This increase in NKG2C expressing cells was reproducible with the NKG2C and DAP12 iNK having a significantly higher NKG2C⁺ population compared to the non-transduced and NKG2C without DAP12 (65.32% v 10.86 and 45.52, p>0.01 and 0.001) (FIG. 4B) and DAP12 was increased as well (FIG. 8). The NKG2C and DAP12 iNK cells also expressed more NKG2C per cell, as shown by MFI, compared to the NKG2C without DAP12 iNK (FIG. 4C). While the NKG2C with DAP12 did not express intracellular markers of adaptive NK cells (PLZF, EAT2 or FcεRIγ), they did express slightly more DAP12 associated receptors NKp44 and KIRs at a higher density (FIG. 9).

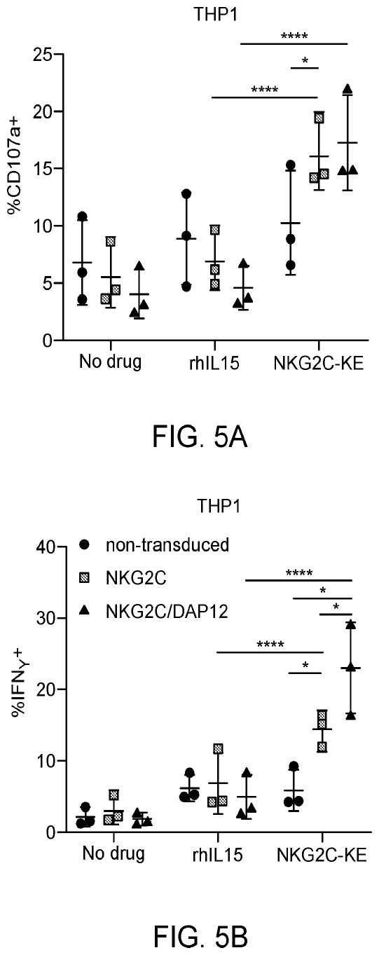

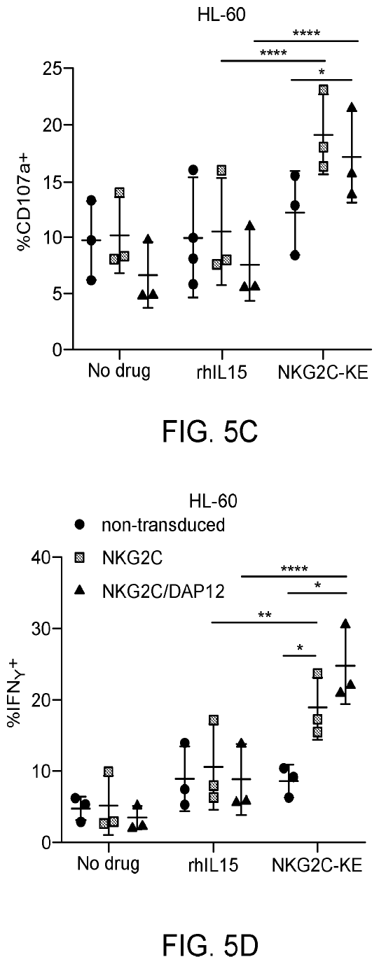

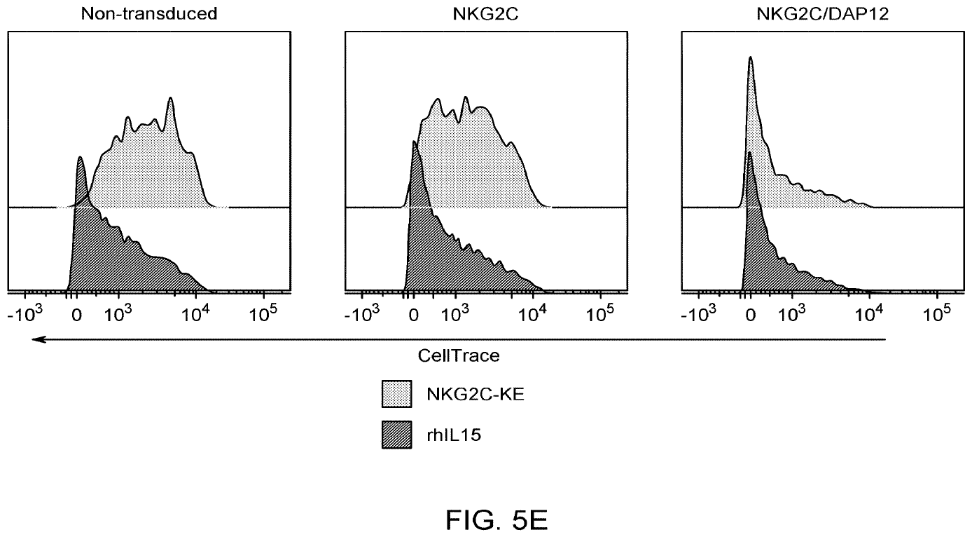

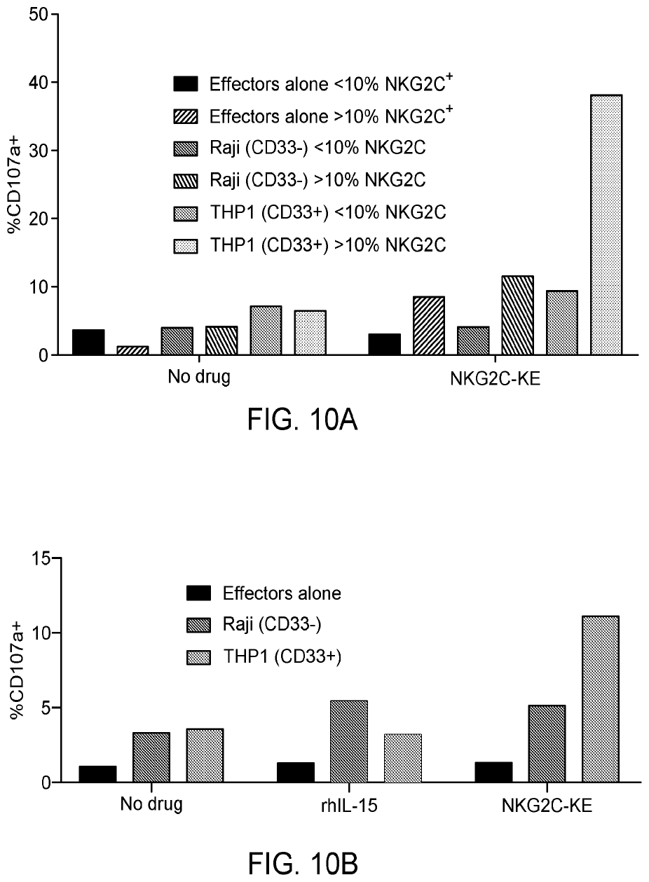

To test the combined therapeutic potential of the NKG2C-KE and NKG2C gene-edited iNK cells, two AML cell lines that highly express CD33 (HL-60 and THP-1) were used as targets in a series of functional assays. Non-transduced and gene-edited iNK cells were co-cultured with targets alone, with rhIL-15, or with the NKG2C-KE and evaluated for degranulation and cytokine production (FIGS. 5A-D). In the transduced iNK, the NKG2C-KE induced significantly more degranulation against THP-1 targets compared to rhIL-15 (NKG2C: 16.1% vs 6.9%, p<0.0001; NKG2C/DAP12: 17.2% vs 4.6%, p<0.0001) and more IFNγ production (NKG2C: 14.4% vs 6.9%, p=0.003; NKG2C/DAP12: 23.0% vs 4.9%, p<0.0001) (FIGS. 5A and 5B). Incubation with CD33⁻ targets did not result in increased degranulation or IFNγ (FIG. 10). The functional responses of non-transduced cells were not affected by the NKG2C-KE, suggesting that low frequencies of NKG2C are insufficient to generate an enhanced response. A similar result was seen with HL-60 cells (FIGS. 5C and 5D). While the gene-edited iNK cells (NKG2C+/−DAP12) exhibited similar levels of degranulation against both tumor cell lines, iNK cells co-transduced cells with both NKG2C and DAP12 produced significantly more IFNγ than NKG2C-transduced cells without DAP12 against the THP-1 cells, but not the HL-60 cells. Stimulation with rhIL-15 induced a strong proliferative signal in both transduced and non-transduced iNK, but the NKG2C-KE mediated more proliferation for iNK cells co-transduced with both NKG2C and DAP12 (FIG. 5E). The enhanced degranulation, IFNγ production, and proliferative response indicate an important role for DAP12 in NKG2C-mediated functions in iNK cell products. Thus, iNK cells engineered with both NKG2C and DAP12 were used in further experiments.

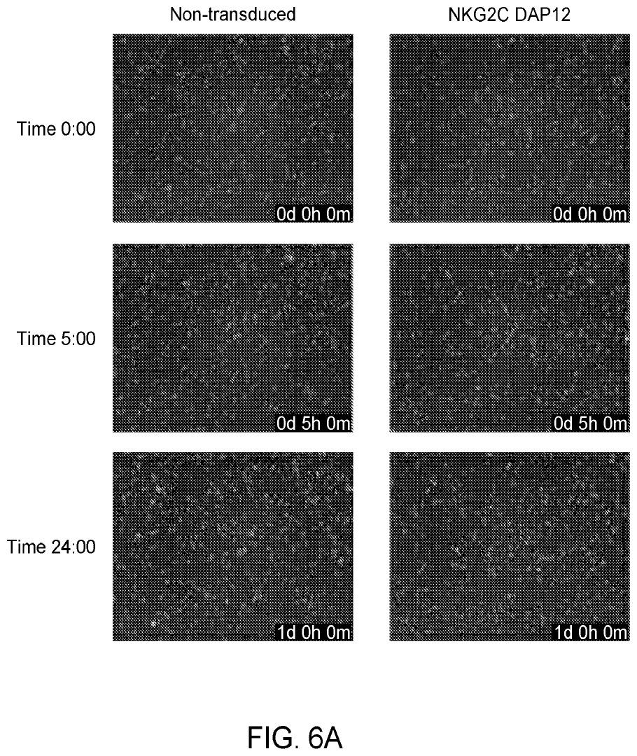

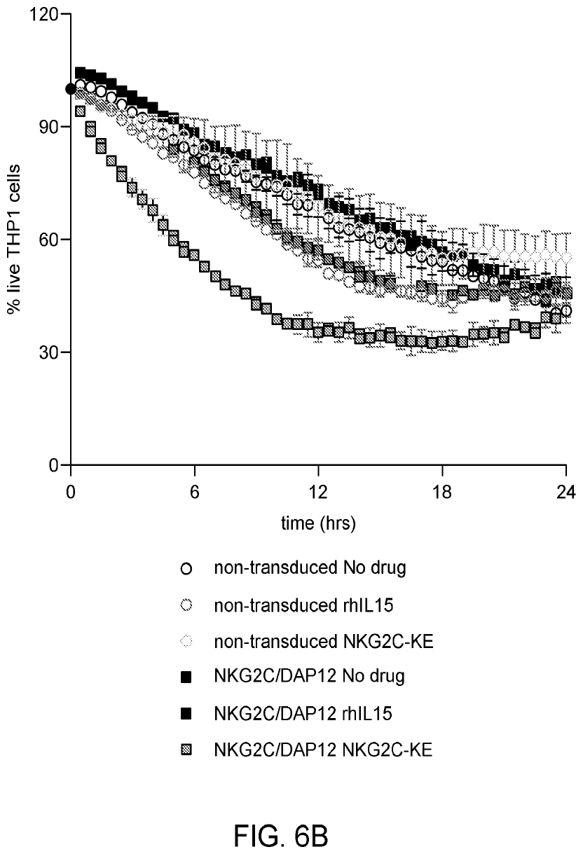

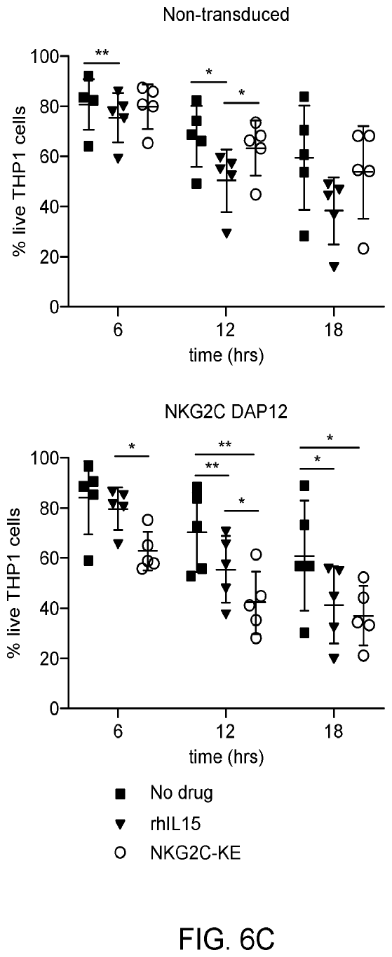

To directly evaluate tumor killing, a dynamic in vitro system was utilized where the loss of fluorescently labeled target cells, upon cell death, can be quantified continuously over a 24-hour period by live imaging. Non-transduced and NKG2C/DAP12-transduced iNK cells were co-cultured with THP-1 targets with and without rhIL-15 or NKG2C-KE and imaged every 30 minutes (FIGS. 6A and 6B). Non-transduced iNK cells exhibited modest natural cytotoxicity against THP-1 cells, which remained largely unchanged by the addition of rhIL-15, though statistically different, or the NKG2C-KE. In contrast, addition of the NKG2C-KE to NKG2C/DAP12-transduced iNK cells resulted in increased killing kinetics in the first 18 hours of exposure with statistical significance consistently at 6 and 12 hours (FIG. 6C).

NKG2C/DAP12-Transduced iNK Cells Targeted with the NKG2C-KE Mediate Cytotoxicity Against Primary AML

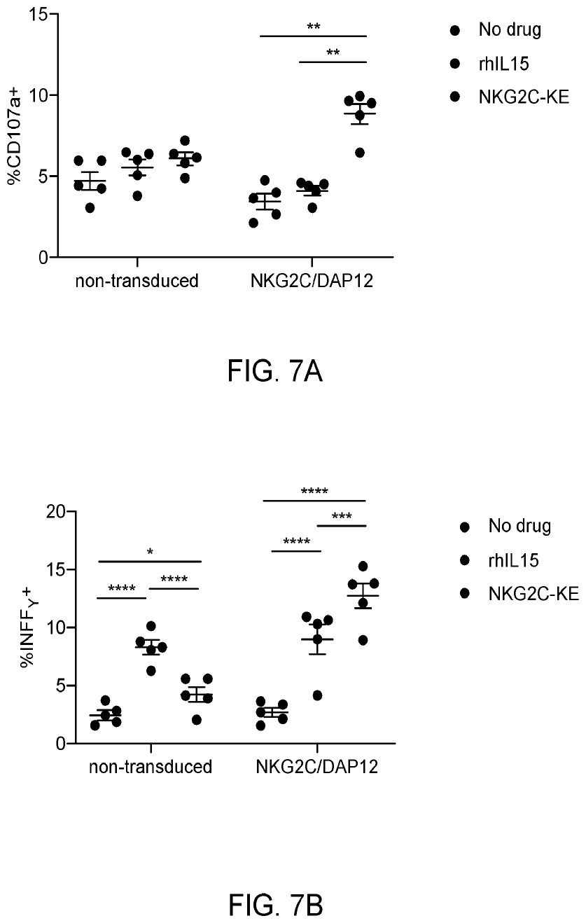

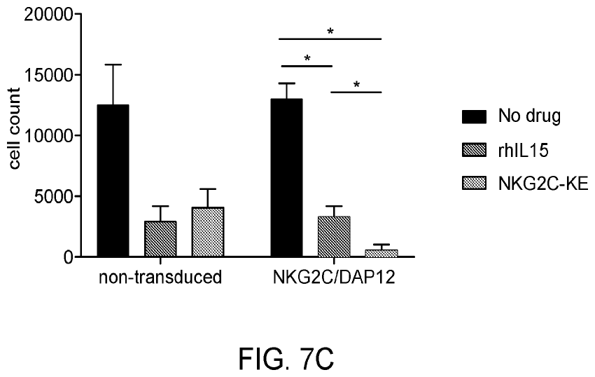

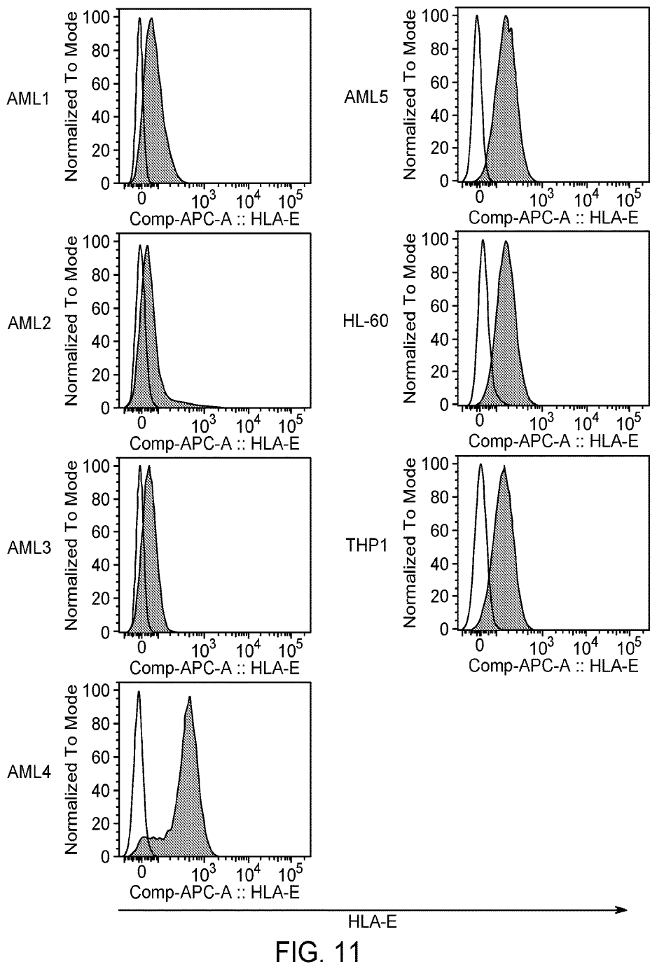

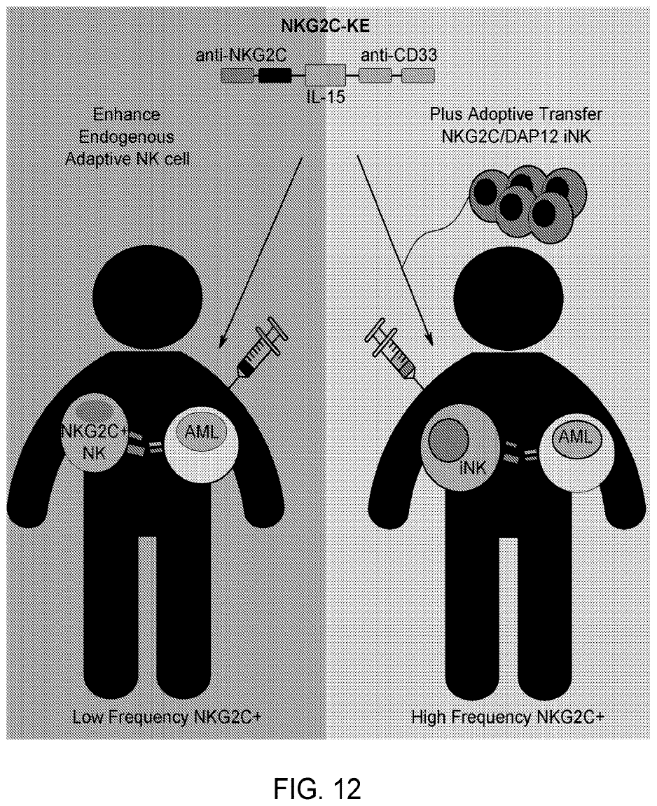

The functional assays described above tested targeting of AML cell lines. To test the functional capacity of NKG2C/DAP12 iNK cells activated with the NKG2C-KE against a more physiologic target, additional function experiments were performed using primary AML blasts. Five AML patient samples, containing 47-97% CD33⁺ blasts were used as targets in a flow cytometry-based functional assay and all expressing HLA-E (FIG. 11). The non-transduced iNK cells exhibited low levels of degranulation against the blasts, whether treated with or without rhIL-15 or the NKG2C-KE (FIG. 7A). Similar functional responses were observed for NKG2C/DAP12 iNK cells in the presence or absence of rhIL-15, even though rhIL-15 has a stronger IL-15 signal than NKG2C-KE. However, when combined with the NKG2C-KE, the NKG2C/DAP12 iNK cells exhibited 2-fold greater degranulation against primary AML blasts. While rhIL-15 potentiated IFNγ production by both iNK lines, the greatest induction was observed with the NKG2C/DAP12-transduced cells targeted with the NKG2C-KE (FIG. 7B). Killing of primary AML blasts was also evaluated by determination of viable AML cell counts after 2 days of co-culture (FIG. 7C). No difference in AML cell numbers was observed when comparing non-transduced iNK cell co-cultures and NKG2C/DAP12-transduced iNK cell co-cultures in the absence of rhIL-15 or the NKG2C-KE. rhIL-15 alone induced a strong natural cytotoxic response from both iNK cell lines, resulting in substantial AML killing. Directing NKG2C/DAP12-transduced iNK cells with the NKG2C-KE to the CD33 antigen resulted in the robust and nearly complete elimination of primary AML targets, (rhIL-15, 3298; NKG2C-KE, 486.7; p=0.02).

Together, these results demonstrate that cell engagers that can bind to a NKG2C polypeptide and can bind a CD33 polypeptide can direct NK cells to CD33⁺ cancer cells and induce an immune response against those CD33⁺ cancer cells. Also as demonstrated herein, these cell engagers can be used to a treat a mammal (e.g., a human) having cancer (e.g., a leukemia such as AML).

### Materials and Methods

### Protein Production

The final construct of the NKG2C-KE was spliced into the Minicircle plasmid (SBI: MN502A-1) with a CMV promoter into the multicloning site. The NKG2C-KE portion contains a start codon, export sequence, an anti-NKG2C scFv, an 18 amino acid sequence flanking wildtype IL-15, an anti-CD33 scFv and a 10×His-Tag. The NKG2C-KE plasmid was transfected into Expi293F (Thermofisher, Waltham, MA: A14527) cells using Expifectimine (Thermofisher: A14524). Supernatant was harvested at day 4-5 post-transfection when cell viability dropped below 80%. Supernatant was then incubated with HisPur cobolt resin (ThermoFisher: 89965) for 1 hour. Resin was washed 3 times then the NKG2C-KE was eluted from HisPur resin through a column using 250 mM imidazole. Protein was desalted using a PD-10 column (GE healthcare, Chicago, IL). Protein was run on a tris-based gel and purity was assessed using GelCode Blue (Thermofisher; 24592) stain. The 161533 TriKE was made as described elsewhere (Hermanson et al., Stem Cells, 34:93-101 (2016)). Briefly, plasmid was transformed into Escherichia coli strain BL21 (DE3) (EMD) then harvested 2 hours later by pelleting. Then bacteria were resuspended and inclusion bodies harvested, then washed to remove endotoxin. Protein was then refolded and purified using FPLC ion exchange chromatography.

### Healthy Donors and Patient Samples

Healthy donor blood was processed to isolate peripheral blood mononuclear cells (PBMCs) using density gradient Ficoll-Paque (GE Healthcare). PBMCs were either cryopreserved in liquid nitrogen or used fresh. AML blasts were obtained and cryopreserved from an apheresis of a de novo (no prior chemotherapy) AML patient with normal cytogenetics and 45-93% AML blast frequencies. All cells were cultured in RPMI-1640 (Thermofisher) with 10% heat inactivated fetal bovine serum and Pen/Strep supplementation at 37° C. and 5% CO₂. PBMCs and AML samples were thawed and rested overnight before use.

### Generation of Expanded NK Cells

Human PBMCs obtained from above protocol were enriched for NK cells (Stemcell). Then cells were cultured for 14 days with feeder cells of irradiated K562 with 41BBL and membrane bound IL-21 in RPMI with 10% heat inactivated FBS, PenStrep and 50 IU/mL IL2. Media was changed every 2-3 day. Feeder cells were added twice at day 0 and day 7.

Generation of NKG2C⁺ NK Cells from iPSCs

Human iPSC culture and differentiation to iCD34⁺ and iNK cells were performed as described elsewhere Cichocki et al., Sci. Transl. Med., 12:eaaz5618 (2020)). At the beginning of the iNK cell differentiation culture, iCD34 cells were plated on stroma cells in B0 media supplemented with cytokines that support NK cell differentiation from hematopoietic progenitors). After iNK cell specification, iNK cells were harvested and co-cultured with modified K562 in supplemented B0 media for expansion. K562 cells were propagated in RPMI 1640 media (ThermoFisher) containing 10% FBS (Hyclone).

### Cell Lines, Antibodies, and Reagents

THP-1 and HL-60 cells were cultured in RPMI-1640 (Gibco) with 10% heat inactivated fetal bovine serum and Pen/Strep supplementation. THP-1 and HL-60 cells were cultured to a density between 0.2-2 million cells/mL. Cell lines were purchased from ATCC. Fluorochrome-conjugated antibodies were purchased from BioLegend (San Diego, CA): anti-CD56 (clone HCD56), anti-IFNγ (clone XMG1.2), anti-CD45 (clone HI30), anti-CD34 (clone 561), anti-NKp44 (clone P44-8), and anti-KIRs (clones HP-MA4, DX27, and DX9), anti-NKG2D (clone 1D11), anti-HLA-E (clone 3D12); BD Biosciences (San Jose, CA): anti-CD3 (clone UCHT1); ThermoFisher (Waltham, MA): Live/Dead Aqua (product #L34966), CellTrace Violet (C34557), Live/Dead Near IR (L34976), CellTrace FarRed (C34564); R&D Systems (Minneapolis, MN): anti-NKG2C (clone 134591), anti-PLZF (clone 6318100), anti-DAP12 (clone 406288); Millipore (Burlington, MA): anti-FceRIγ (polyclonal); proteintech (Rosemont, IL): anti-EAT2 (SH2D1B) (polyclonal); Beckman Coulter (Indianapolis, Indiana): anti-NKG2A (clone z199); Sartorius (France): Caspase 3/7 apoptosis assay reagent FITC (Satorius 4440).

### NK Cell Function Assays

Effectors were incubated with targets with or without NKG2C-KE at a 2:1 effector:target (E:T) ratio. Anti-CD107a antibody was added at the beginning of co-culture. One hour into the incubation, GolgiStop and GolgiPlug were added to each well and incubated for an additional 4 hours. At the end of the 5-hour incubation, cells were stained with Live/Dead Aqua before being surface stained for CD3 and CD56. Cells were then fixed with 2% paraformaldehyde in PBS for 20 minutes and permeabilized with 0.1% Triton X for 5 minutes. followed by intracellular staining for IFNγ. Samples were analyzed on an LSRII Flow Cytometer (BD) and analyzed with FlowJo software (BD, Ashland, OR). For analysis of proliferation, cells were stained with CellTrace Violet and then incubated with NKG2C-KE or recombinant human IL-15 (R&D Systems, Minneapolis, MN) for 7 days at 37° C., 5% CO₂. At the end of 7 days, cells were washed and stained with Live/Dead Near IR and then surface stained with antibodies against CD3, CD56 and NKG2C. For live imaging, THP-1 cells were stained with CellTrace Far Red prior to plating. IncuCyte Caspase 3/7 apoptosis assay reagent was added to each well. iNK cells were added at 5:1 E:T ratio. Plates were then placed in Incucyte S3 (Satorus Inc., France) for 24 hours. Images were taken every 30 minutes. Graphs were created using values for live THP-1 cell counts normalized to no effector control values and to initial plated cell counts. All conditions were run in triplicate. Primary AML cells were stained with CellTrace Violet and then co-incubated with iNK cells and NKG2C-KE for 2 days. Cells were then stained with antibodies against CD45 and CD34 and analyzed by flow cytometry. Remaining AML cells with a CellTrace⁺CD45^{intermediate} CD34⁺ cells were counted.

### In Vivo Mouse Study

The HL60-Luc model was as described elsewhere (Dezell et al., Biol. Blood Marrow Transplant., 18: 536-545 (2012); Miller el al., Blood, 83: 2594-2601 (1994); and Hermanson et al., Stem Cells, 34:93-101 (2016)). Briefly, female NOD-SCID-gamma (NSG) mice were injected with 750,000 HL60-luc intravenously. Then three days later, bioluminescence imaging (BLI) was performed on mice and mice were divided into groups with equal tumor load. Mice were injected with 5 million expanded NK cells intravenously and drug treatments were started at 5 times weekly for 3 weeks. Treatments were injected intraperitoneally. BLI was then measured at day 6, 13, 27 and 35 and facial vein bleeding was done at day 14 and 28. Red blood cells were lysed and then stained for hCD45, mCD45, CD3, CD56, NKG2C and CD16.

### Statistics

GraphPad Prism (GraphPad Prism Software, Inc, La Jolla, CA) was used to plot graphs with error bars showing mean±SEM or ±SD where appropriate and correlation curves with 95% CI. GraphPad was also used to calculate linear regression t-tests, one-way ANOVA and two-way ANOVA with and without repeated measurement (RM) analysis where appropriate and determine statistical significance as *P<0.05, **P<0.01, ***P<0.001, and ****P<0.0001. Specific statistical analysis used for each graph is indicated in figure legends.

### Example 2: Exemplary NKG2C Polypeptides

This Example provides an amino acid sequence of a human NKG2C polypeptide (SEQ ID NO:1). The underlined and bolded amino acid sequence of this human NKG2C polypeptide depicts the NKG2C extracellular domain (SEQ ID NO:2).

MNKQRGTFSEVSLAQDPKRQQRKPKGNKSSISGTEQEIFQVELNL
QNPSLNHQGIDKIYDCQGLLPPPEKLTAEVLGIICIVLMATVLKT
IVLIPFLEQNNESPNTRTQKARHCGHCPEEWITYSNSCYYIGKER
RTWEESLLACTSKNSSLLSIDNEEEMKFLASILPSSWIGVFRNSS
HHPWVTINGLAFKHKIKDSDNAELNCAVLQVNRLKSAQCGSSMIY
HCKHKL

### Example 3: Exemplary scFvs Having the Ability to Bind a NKG2C Polypeptide

This Example provides the amino acid sequences of the heavy chain variable domain and the light chain variable domain of exemplary scFvs. The CDRs, framework sequences, and constant domains of each also are provided and delineated.

Anti-NKG2C VH (with the CDRs underlined):
(SEQ ID NO: 19)
EVQLQQSGAELVKPGASVTLSCTASGFNIKDTYMHWVQQRPEQGL
EWIGRIDPENGYTKYDPNFQGKATITADTSSNTAYLQLSSLTSED
TAVYHCARSRTLFWYFDVWGAGTTVTVSS
Framework Region 1 of heavy chain variable domain:
(SEQ ID NO: 11)
EVQLQQSGAELVKPGASVTLSCTAS
CDR1 of heavy chain variable domain:
(SEQ ID NO: 5)
GFNIKDT
Framework Region 2 of heavy chain variable domain:
(SEQ ID NO: 12)
YMHWVQQRPEQGLEWIGRI
CDR2 of heavy chain variable domain:
(SEQ ID NO: 6)
DPENGY
Framework Region 3 of heavy chain variable domain:
(SEQ ID NO: 13)
TKYDPNFQGKATITADTSSNTAYLQLSSLTSEDTAVYHCAR
CDR3 of heavy chain variable domain:
(SEQ ID NO: 7)
SRTLFWYFDV
Framework Region 4 of heavy chain variable domain:
(SEQ ID NO: 14)
WGAGTTVTVSS
Anti-NKG2C VL (with the CDRs underlined):
(SEQ ID NO: 20)
NIMMTQSPSSLAVSAGEKVTMSCKSSQSVLYSSNQKNYLAWYQQK
PGQSPKLLIYWASTRESGVPDRFTGSGSGTDFTLTITNIQAEDLA
VYYCHQYLSSYTFGGGTKLEIKRA
Framework Region 1 of light chain variable domain:
(SEQ ID NO: 15)
NIMMTQSPSSLAVSAGEKVTMSC
CDR1 of light chain variable domain:
(SEQ ID NO: 8)
KSSQSVLYSSNQKNYLA
Framework Region 2 of light chain variable domain:
(SEQ ID NO: 16)
WYQQKPGQSPKLLIY
CDR2 of light chain variable domain:
(SEQ ID NO: 9)
WASTRES
Framework Region 3 of light chain variable domain:
(SEQ ID NO: 17)
GVPDRFTGSGSGTDFTLTITNIQAEDLAVYYC
CDR3 of light chain variable domain:
(SEQ ID NO: 10)
HQYLSSYT
Framework Region 4 of light chain variable domain:
(SEQ ID NO: 18)
FGGGTKLEIKRA

### Example 4: Nucleic Acids Encoding Exemplary NKG2C scFvs

This Example provides the nucleic acid sequences encoding the exemplary NKG2C scFv indicated in Example 3.

Nucleic acid encoding SEQ ID NO: 19
(NKG2C scFv heavy chain):
(SEQ ID NO: 42)
GAGGTACAGCTGCAGCAGTCTGGAGCCGAGCTGGTCAAACCCGGC
GCTTCTGTCACTCTGAGCTGCACCGCATCTGGGTTCAACATTAAG
GACACATACATGCACTGGGTTCAGCAGCGCCCAGAGCAGGGACTG
GAATGGATTGGCAGAATCGACCCCGAAAACGGATACACGAAGTAT
GACCCCAATTTCCAGGGAAAGGCAACCATCACGGCTGATACTTCC
TCAAACACCGCATATTTGCAACTGTCATCACTGACCAGTGAGGAT
ACTGCTGTATATCACTGTGCTCGCAGCCGTACCCTGTTCTGGTAT
TTCGACGTCTGGGGTGCCGGCACAACTGTTACTGTTAGTTCG
Nucleic acid encoding SEQ ID NO: 20
(NKG2C scFv light chain):
(SEQ ID NO: 43)
AACATAATGATGACCCAGTCGCCTTCTTCCCTAGCCGTGAGCGCA
GGCGAAAAGGTCACAATGAGTTGCAAATCAAGCCAGTCCGTCCTG
TACTCGAGCAACCAGAAAAATTACCTCGCATGGTATCAACAGAAG
CCTGGACAATCACCTAAGCTGCTGATATATTGGGCTTCCACACGC
GAGTCTGGTGTTCCCGACCGATTTACTGGGTCCGGGAGCGGCACA
GACTTTACCTTGACAATCACCAATATCCAGGCCGAGGACCTAGCT
GTATATTATTGTCACCAGTATCTTTCTAGTTATACTTTCGGCGGA
GGGACTAAACTCGAGATTAAGAGAGCC

### Example 5: Exemplary CD33 Polypeptides

This Example provides an amino acid sequence of a human CD33 polypeptide (SEQ ID NO:3). The underlined and bolded amino acid sequence of this human CD33 polypeptide depicts the CD33 polypeptide extracellular domain (SEQ ID NO:4).

DPNFWLQVQESVTVQEGLCVLVPCTFFHPIPYYDKNSPVHGYWFR
EGAIISRDSPVATNKLDQEVQEETQGRFRLLGDPSRNNCSLSIVD
ARRRDNGSYFFRMERGSTKYSYKSPQLSVHVTDLTHRPKILIPGT
LEPGHSKNLTCSVSWACEQGTPPIFSWLSAAPTSLGPRTTHSSVL
IITPRPQDHGTNLTCQVKFAGAGVTTERTIQLNVTYVPQNPTTGI
FPGDGSGKQETRAGVVHGAIGGAGVTALLALCLCLIFFIVKTHRR
KAARTAVGRNDTHPTTGSASPKHQKKSKLHGPTETSSCSGAAPTV
EMDEELHYASLNFHGMNPSKDTSTEYSEVRTQ

### Example 6: Exemplary scFv Having the Ability to Bind a CD33 Polypeptide

This Example provides the amino acid sequences of the heavy chain variable domain and the light chain variable domain of an exemplary scFv. The CDRs, framework sequences, and constant domains of each also are provided and delineated.

Anti-CD33 VH (with the CDRs underlined):
(SEQ ID NO: 38)
QVQLVQSGAEVKKPGSSVKVSCKASGYTFTDYNMHWVRQAPG
QGLEWIGYIYPYNGGTGYNQKFKSKATITADESTNTAYMELS
SLRSEDTAVYYCARGRPAMDYWGQGTLVTVSS
Framework Region 1 of heavy chain variable
domain:
(SEQ ID NO: 30)
QVQLVQSGAEVKKPGSSVKVSCKAS
CDR1 of heavy chain variable domain:
(SEQ ID NO: 24)
GYTFTDY
Framework Region 2 of heavy chain variable
domain:
(SEQ ID NO: 31)
NMHWVRQAPGQGLEWIGYI
CDR2 of heavy chain variable domain:
(SEQ ID NO: 25)
YPYNGG
Framework Region 3 of heavy chain
variable domain:
(SEQ ID NO: 32)
TGYNQKFKSKATITADESTNTAYMELSSLRSEDTAVYYCAR
CDR3 of heavy chain variable domain:
(SEQ ID NO: 26)
GRPAMDY
Framework Region 4 of heavy chain
variable domain:
(SEQ ID NO: 33)
WGQGTLVTVSS
Anti-CD33 VL (with the CDRs underlined):
(SEQ ID NO: 39)
DIQMTQSPSSLSASVGDRVTITCRASESVDNYGISFMNW
FQQKPGKAPKLLIYAASNQGSGVPSRFSGSGSGTDFTLT
ISSLQPDDFATYYCQQSKEVPWTFGQGTKVEIK
Framework Region 1 of light chain variable
domain:
(SEQ ID NO: 34)
DIQMTQSPSSLSASVGDRVTITC
CDR1 of light chain variable domain:
(SEQ ID NO: 27)
RASESVDNYGISFMN
Framework Region 2 of light chain variable
domain:
(SEQ ID NO: 35)
WFQQKPGKAPKLLIY
CDR2 of light chain variable domain:
(SEQ ID NO: 28)
AASNQGS
Framework Region 3 of light chain variable
domain:
(SEQ ID NO: 36)
GVPSRFSGSGSGTDFTLTISSLQPDDFATYYC
CDR3 of light chain variable domain:
(SEQ ID NO: 29)
QQSKEVPWT
Framework Region 4 of light chain variable
domain:
(SEQ ID NO: 37)
FGQGTKVEIK

### Example 7. Nucleic Acids Encoding Exemplary CD33 scFv

This Example provides the nucleic acid sequences encoding the exemplary CD33 scFv indicated in Example 6.

Nucleic acid encoding SEQ ID NO: 38
(CD33 scFv heavy chain):
(SEQ ID NO: 44)
CAGGTGCAACTGGTTCAATCTGGCGCCGAGGTAAAAAAACCAGGC
TCATCTGTAAAAGTGAGCTGTAAGGCTTCCGGATACACTTTCACT
GACTACAACATGCATTGGGTTAGGCAAGCACCCGGACAAGGACTC
GAGTGGATCGGGTACATATACCCTTACAACGGGGGAACAGGGTAC
AATCAGAAGTTCAAAAGTAAGGCTACTATTACAGCCGATGAGAGC
ACTAACACCGCCTACATGGAGCTTAGCAGTCTGAGATCTGAAGAT
ACCGCCGTGTACTATTGCGCACGGGGCAGACCCGCCATGGATTAC
TGGGGCCAGGGCACCCTGGTTACGGTGTCTAGT
Nucleic acid encoding SEQ ID NO: 39
(CD33 scFv light chain):
(SEQ ID NO: 45)
GACATTCAGATGACTCAGTCCCCCAGCTCACTGTCTGCTTCCGTG
GGCGACCGCGTGACTATTACGTGTCGCGCCTCTGAATCAGTGGAC
AATTATGGCATATCCTTCATGAACTGGTTCCAGCAGAAACCAGGC
AAGGCTCCCAAGCTGCTTATATACGCGGCGTCTAATCAAGGCAGT
GGTGTGCCTTCCCGATTCAGTGGTTCAGGGAGTGGGACTGATTTC
ACTCTGACAATTTCAAGCCTCCAGCCAGATGATTTCGCTACATAC
TACTGTCAACAGTCTAAGGAAGTGCCATGGACATTCGGGCAGGGT
ACCAAGGTGGAGATCAAG

### Example 8: Exemplary Cell Engager

This Example provides the amino acid sequence of an anti-NKG2C cell engager that also can bind a CD33 polypeptide and includes an IL-15 polypeptide. The various components of this cell engager (e.g., domains and linkers) are provided and delineated.

(SEQ ID NO: 46)
EVQLQQSGAELVKPGASVTLSCTASGFNIKDTYMHWVQQRPEQGL
EWIGRIDPENGYTKYDPNFQGKATITADTSSNTAYLQLSSLTSED
TAVYHCARSRTLFWYFDVWGAGTTVTVSSGGGGSGGGGSGGGGSN
IMMTQSPSSLAVSAGEKVTMSCKSSQSVLYSSNQKNYLAWYQQKP
GQSPKLLIYWASTRESGVPDRFTGSGSGTDFTLTITNIQAEDLAV
YYCHQYLSSYTFGGGTKLEIKRAGSTSGSGKPGSGEGSTKGNWVN
VISDLKKIEDLIQSMHIDATLYTESDVHPSCKVTAMKCFLLELQV
ISLESGDASIHDTVENLIILANNSLSSNGNVTESGCKECEELEEK
NIKEFLQSFVHIVQMFINTSGSTSGSGKPGSGEGSTKGQVQLVQS
GAEVKKPGSSVKVSCKASGYTFTDYNMHWVRQAPGQGLEWIGYIY
PYNGGTGYNQKFKSKATITADESTNTAYMELSSLRSEDTAVYYCA
RGRPAMDYWGQGTLVTVSSGGGGSGGGGSGGGGSDIQMTQSPSSL
SASVGDRVTITCRASESVDNYGISFMNWFQQKPGKAPKLLIYAAS
NQGSGVPSRFSGSGSGTDFTLTISSLQPDDFATYYCQQSKEVPWT
FGQGTKVEIKVDEHHHHHHHHHH
BLK Alb signal polypeptides:
(SEQ ID NO: 47)
KWVTFISLLFLFSSAYS
anti-NKG2C VH (with the CDRs underlined):
(SEQ ID NO: 19)
EVQLQQSGAELVKPGASVTLSCTASGFNIKDTYMHWVQQRPEQGL
EWIGRIDPENGYTKYDPNFQGKATITADTSSNTAYLQLSSLTSED
TAVYHCARSRTLFWYFDVWGAGTTVTVSS
Linker:
(SEQ ID NO: 21)
GGGGSGGGGSGGGGS
NKG2C VL (with the CDRs underlined):
(SEQ ID NO: 20)
NIMMTQSPSSLAVSAGEKVTMSCKSSQSVLYSSNQKNYLAWYQQK
PGQSPKLLIYWASTRESGVPDRFTGSGSGTDFTLTITNIQAEDLA
VYYCHQYLSSYTFGGGTKLEIKRA
Whitlow linker:
(SEQ ID NO: 41)
GSTSGSGKPGSGEGSTKG
IL-15 polypeptide:
(SEQ ID NO: 40)
NWVNVISDLKKIEDLIQSMHIDATLYTESDVHPSCKVTAMKCFLL
ELQVISLESGDASIHDTVENLIILANNSLSSNGNVTESGCKECEE
LEEKNIKEFLQSFVHIVQMFINTS
Anti-CD33 VH (with the CDRs underlined):
(SEQ ID NO: 38)
QVQLVQSGAEVKKPGSSVKVSCKASGYTFTDYNMHWVRQAPGQGL
EWIGYIYPYNGGTGYNQKFKSKATITADESTNTAYMELSSLRSED
TAVYYCARGRPAMDYWGQGTLVTVSS
Linker:
(SEQ ID NO: 21)
GGGGSGGGGSGGGGS
Anti-CD33 VL (with the CDRs underlined):
(SEQ ID NO: 39)
DIQMTQSPSSLSASVGDRVTITCRASESVDNYGISEMNWFQQKPG
KAPKLLIYAASNQGSGVPSRFSGSGSGTDFTLTISSLQPDDFATY
YCQQSKEVPWTFGQGTKVEIK
Spacer:
VDE
10x His polypeptide tag:
(SEQ ID NO: 48)
HHHHHHHHHH

### Example 9. Nucleic Acids Encoding Exemplary Anti-NKG2C Cell Engager

This Example provides the nucleic acid sequences encoding the exemplary NKG2C cell engager indicated in Example 5. The nucleic acid sequences encoding various components of this cell engager (e.g., domains and linkers) are provided and delineated.

(SEQ ID NO: 49)
GCCGCCACCATGAAGTGGGTAACCTTTATTTCCCTTCTTTTTCTC
TTTAGCTCGGCTTATTCCGAGGTACAGCTGCAGCAGTCTGGAGCC
GAGCTGGTCAAACCCGGCGCTTCTGTCACTCTGAGCTGCACCGCA
TCTGGGTTCAACATTAAGGACACATACATGCACTGGGTTCAGCAG
CGCCCAGAGCAGGGACTGGAATGGATTGGCAGAATCGACCCCGAA
AACGGATACACGAAGTATGACCCCAATTTCCAGGGAAAGGCAACC
ATCACGGCTGATACTTCCTCAAACACCGCATATTTGCAACTGTCA
TCACTGACCAGTGAGGATACTGCTGTATATCACTGTGCTCGCAGC
CGTACCCTGTTCTGGTATTTCGACGTCTGGGGTGCCGGCACAACT
GTTACTGTTAGTTCGGGCGGTGGCGGCTCTGGTGGTGGCGGTAGT
GGCGGAGGTGGTAGCAACATAATGATGACCCAGTCGCCTTCTTCC
CTAGCCGTGAGCGCAGGCGAAAAGGTCACAATGAGTTGCAAATCA
AGCCAGTCCGTCCTGTACTCGAGCAACCAGAAAAATTACCTCGCA
TGGTATCAACAGAAGCCTGGACAATCACCTAAGCTGCTGATATAT
TGGGCTTCCACACGCGAGTCTGGTGTTCCCGACCGATTTACTGGG
TCCGGGAGCGGCACAGACTTTACCTTGACAATCACCAATATCCAG
GCCGAGGACCTAGCTGTATATTATTGTCACCAGTATCTTTCTAGT
TATACTTTCGGCGGAGGGACTAAACTCGAGATTAAGAGAGCCGGC
AGTACCAGCGGGTCAGGGAAACCTGGCAGTGGGGAAGGTTCCACA
AAAGGTAACTGGGTGAATGTAATAAGTGATTTGAAAAAAATTGAA
GATCTTATTCAATCTATGCATATTGATGCTACTTTATATACGGAA
AGTGATGTTCACCCCAGTTGCAAAGTAACAGCAATGAAGTGCTTT
CTCTTGGAGTTACAAGTTATTTCACTTGAGTCCGGAGATGCAAGT
ATTCATGATACAGTAGAAAATCTGATCATCCTAGCAAACAACAGT
TTGTCTTCTAATGGGAATGTAACAGAATCTGGATGCAAAGAATGT
GAGGAACTGGAGGAAAAAAATATTAAAGAATTTTTGCAGAGTTTT
GTACATATTGTCCAAATGTTCATCAACACTTCTGGCAGTACCAGC
GGGTCAGGGAAACCTGGCAGTGGGGAAGGTTCCACAAAAGGTCAG
GTGCAACTGGTTCAATCTGGCGCCGAGGTAAAAAAACCAGGCTCA
TCTGTAAAAGTGAGCTGTAAGGCTTCCGGATACACTTTCACTGAC
TACAACATGCATTGGGTTAGGCAAGCACCCGGACAAGGACTCGAG
TGGATCGGGTACATATACCCTTACAACGGGGGAACAGGGTACAAT
CAGAAGTTCAAAAGTAAGGCTACTATTACAGCCGATGAGAGCACT
AACACCGCCTACATGGAGCTTAGCAGTCTGAGATCTGAAGATACC
GCCGTGTACTATTGCGCACGGGGCAGACCCGCCATGGATTACTGG
GGCCAGGGCACCCTGGTTACGGTGTCTAGTGGCGGAGGTGGGAGC
GGTGGGGGGGGTAGCGGGGGAGGCGGCTCTGACATTCAGATGACT
CAGTCCCCCAGCTCACTGTCTGCTTCCGTGGGCGACCGCGTGACT
ATTACGTGTCGCGCCTCTGAATCAGTGGACAATTATGGCATATCC
TTCATGAACTGGTTCCAGCAGAAACCAGGCAAGGCTCCCAAGCTG
CTTATATACGCGGCGTCTAATCAAGGCAGTGGTGTGCCTTCCCGA
TTCAGTGGTTCAGGGAGTGGGACTGATTTCACTCTGACAATTTCA
AGCCTCCAGCCAGATGATTTCGCTACATACTACTGTCAACAGTCT
AAGGAAGTGCCATGGACATTCGGGCAGGGTACCAAGGTGGAGATC
AAGGTCGACGAGCATCATCATCATCACCACCACCACCACCACTGA
Kozak sequence:
GCCGCCACC
Start codon:
ATG
nucleic acid sequence encoding a BLK Alb signal
polypeptides:
(SEQ ID NO: 50)
AAGTGGGTAACCTTTATTTCCCTTCTTTTTCTCTTTAGCTCGGCT
TATTCC
nucleic acid sequence encoding an anti-NKG2C
VH:
(SEQ ID NO: 51)
GAGGTACAGCTGCAGCAGTCTGGAGCCGAGCTGGTCAAACCCGGC
GCTTCTGTCACTCTGAGCTGCACCGCATCTGGGTTCAACATTAAG
GACACATACATGCACTGGGTTCAGCAGCGCCCAGAGCAGGGACTG
GAATGGATTGGCAGAATCGACCCCGAAAACGGATACACGAAGTAT
GACCCCAATTTCCAGGGAAAGGCAACCATCACGGCTGATACTTCC
TCAAACACCGCATATTTGCAACTGTCATCACTGACCAGTGAGGAT
ACTGCTGTATATCACTGTGCTCGCAGCCGTACCCTGTTCTGGTAT
TTCGACGTCTGGGGTGCCGGCACAACTGTTACTGTTAGTTCG
nucleic acid sequence encoding a linker:
(SEQ ID NO: 52)
GGCGGTGGCGGCTCTGGTGGTGGCGGTAGTGGCGGAGGTGGTAGC
nucleic acid sequence encoding an anti-NKG2C
VL:
(SEQ ID NO: 53)
AACATAATGATGACCCAGTCGCCTTCTTCCCTAGCCGTGAGCGCA
GGCGAAAAGGTCACAATGAGTTGCAAATCAAGCCAGTCCGTCCTG
TACTCGAGCAACCAGAAAAATTACCTCGCATGGTATCAACAGAAG
CCTGGACAATCACCTAAGCTGCTGATATATTGGGCTTCCACACGC
GAGTCTGGTGTTCCCGACCGATTTACTGGGTCCGGGAGCGGCACA
GACTTTACCTTGACAATCACCAATATCCAGGCCGAGGACCTAGCT
GTATATTATTGTCACCAGTATCTTTCTAGTTATACTTTCGGCGGA
GGGACTAAACTCGAGATTAAGAGAGCC
nucleic acid sequence encoding a Whitlow
linker:
(SEQ ID NO: 54)
GGCAGTACCAGCGGGTCAGGGAAACCTGGCAGTGGGGAAGGTTCC
ACAAAAGGT
nucleic acid sequence encoding an IL-15
polypeptide:
(SEQ ID NO: 55)
AACTGGGTGAATGTAATAAGTGATTTGAAAAAAATTGAAGATCTT
ATTCAATCTATGCATATTGATGCTACTTTATATACGGAAAGTGAT
GTTCACCCCAGTTGCAAAGTAACAGCAATGAAGTGCTTTCTCTTG
GAGTTACAAGTTATTTCACTTGAGTCCGGAGATGCAAGTATTCAT
GATACAGTAGAAAATCTGATCATCCTAGCAAACAACAGTTTGTCT
TCTAATGGGAATGTAACAGAATCTGGATGCAAAGAATGTGAGGAA
CTGGAGGAAAAAAATATTAAAGAATTTTTGCAGAGTTTTGTACAT
ATTGTCCAAATGTTCATCAACACTTCT
nucleic acid sequence encoding an anti-CD33
VH:
(SEQ ID NO: 56)
CAGGTGCAACTGGTTCAATCTGGCGCCGAGGTAAAAAAACCAGGC
TCATCTGTAAAAGTGAGCTGTAAGGCTTCCGGATACACTTTCACT
GACTACAACATGCATTGGGTTAGGCAAGCACCCGGACAAGGACTC
GAGTGGATCGGGTACATATACCCTTACAACGGGGGAACAGGGTAC
AATCAGAAGTTCAAAAGTAAGGCTACTATTACAGCCGATGAGAGC
ACTAACACCGCCTACATGGAGCTTAGCAGTCTGAGATCTGAAGAT
ACCGCCGTGTACTATTGCGCACGGGGCAGACCCGCCATGGATTAC
TGGGGCCAGGGCACCCTGGTTACGGTGTCTAGT
nucleic acid sequence encoding a linker:
(SEQ ID NO: 57)
GGCGGAGGTGGGAGCGGTGGGGGGGGTAGCGGGGGAGGCGGCTCT
nucleic acid sequence encoding an anti-CD33 VL:
(SEQ ID NO: 58)
GACATTCAGATGACTCAGTCCCCCAGCTCACTGTCTGCTTCCGTG
GGCGACCGCGTGACTATTACGTGTCGCGCCTCTGAATCAGTGGAC
AATTATGGCATATCCTTCATGAACTGGTTCCAGCAGAAACCAGGC
AAGGCTCCCAAGCTGCTTATATACGCGGCGTCTAATCAAGGCAGT
GGTGTGCCTTCCCGATTCAGTGGTTCAGGGAGTGGGACTGATTTC
ACTCTGACAATTTCAAGCCTCCAGCCAGATGATTTCGCTACATAC
TACTGTCAACAGTCTAAGGAAGTGCCATGGACATTCGGGCAGGGT
ACCAAGGTGGAGATCAAG
nucleic acid sequence encoding a spacer:
GTCGACGAG
nucleic acid sequence encoding a 10x
His polypeptide tag:
(SEQ ID NO: 59)
CATCATCATCATCACCACCACCACCACCAC
Stop codon:
TGA

### Example 10: Nucleic Acids Encoding Exemplary NKG2C Polypeptide

(SEQ ID NO: 60)
CCCTCACATCACACAGCTGCAGAGATGAATAAACAAAGAGGAACC
TTCTCAGAAGTGAGTCTGGCCCAGGACCCAAAGCGGCAGCAAAGG
AAACCTAAAGGCAATAAAAGCTCCATTTCAGGAACCGAACAGGAA
ATATTCCAAGTAGAATTAAATCTTCAAAATCCTTCCCTGAATCAT
CAAGGGATTGATAAAATATATGACTGCCAAGGTTTACTGCCACCT
CCAGAGAAGCTCACTGCCGAGGTCCTAGGAATCATTTGCATTGTC
CTGATGGCCACTGTGTTAAAAACAATAGTTCTTATTCCTTTCCTG
GAGCAGAACAATTTTTCCCCGAATACAAGAACGCAGAAAGCACGT
CATTGTGGCCATTGTCCTGAGGAGTGGATTACATATTCCAACAGT
TGTTATTACATTGGTAAGGAAAGAAGAACTTGGGAAGAGAGTTTG
CTGGCCTGTACTTCGAAGAACTCCAGTCTGCTTTCTATAGATAAT
GAAGAAGAAATGAAATTTCTGGCCAGCATTTTACCTTCCTCATGG
ATTGGTGTGTTTCGTAACAGCAGTCATCATCCATGGGTGACAATA
AATGGTTTGGCTTTCAAACATAAGATAAAAGACTCAGATAATGCT
GAACTTAACTGTGCAGTGCTACAAGTAAATCGACTTAAATCAGCC
CAGTGTGGATCTTCAATGATATATCATTGTAAGCATAAGCTTTAG
AAGTAAAGCATTTGCGTTTGCAGTGCATCAGATACATTTTATATT
TCTTAAAATAGAAATATTATGATTGCATAAATCTGAAAATGAATT
ATGTTATTTGCTCTGATACAAAAATTCTAAATCAATTATTGAAAT
AGGATGCACACAATTACTAAAGTACAGACATCCTAGCATTTGTGT
CGGGCTCATTTTGCTCAACATGGTATTTGTGGTTTTCAGCCTTTC
TAAAAGTTGCATGTTATGTGAGTCAGCTTATAGGAAGTACCAAGA
ACAGTCAAACCCATGGAGACAGAAAGTAGAATAGTGGTTGCCAAT
GTCTCAGGGAGGTTGAAATAGGAGATGACCACTAATTGATAGAAC
GTTTCTTTGTGTCGTGATGAAAACTTTCTAAATTTCAGTAGTGGT
GATGGTTGTAACTCTGCGAATATACTAAACATCATTGATTTTTAA
TCATTTTAAGTGCATGAAATGTATGCTTTGTACATGACACTTCAA
TAAAGCTATCCAGAAAAAAAAAA

### Example 11: Exemplary DAP12 Sequences

Exemplary DAP12 polypeptide sequence
(SEQ ID NO: 61)
GGLEPCSRLLLLPLLLAVSGLRPVQAQAQSDCSCSTVSPGVLAGI
VMGDLVLTVLIALAVYFLGRLVPRGRGAAEAATRKQRITETESPY
QELQGQRSDVYSDLNTQRPYYK
Nucleic acid encoding an exemplary DAP12
polypeptide sequence
(SEQ ID NO: 62)
CCACGCGTCCGCGCTGCGCCACATCCCACCGGCCCTTACACTGTG
GTGTCCAGCAGCATCCGGCTTCATGGGGGGACTTGAACCCTGCAG
CAGGCTCCTGCTCCTGCCTCTCCTGCTGGCTGTAAGTGGTCTCCG
TCCTGTCCAGGCCCAGGCCCAGAGCGATTGCAGTTGCTCTACGGT
GAGCCCGGGCGTGCTGGCAGGGATCGTGATGGGAGACCTGGTGCT
GACAGTGCTCATTGCCCTGGCCGTGTACTTCCTGGGCCGGCTGGT
CCCTCGGGGGCGAGGGGCTGCGGAGGCAGCGACCCGGAAACAGCG
TATCACTGAGACCGAGTCGCCTTATCAGGAGCTCCAGGGTCAGAG
GTCGGATGTCTACAGCGACCTCAACACACAGAGGCCGTATTACAA
ATGAGCCCGAATCATGACAGTCAGCAACATGATACCTGGATCCAG
CCATTCCTGAAGCCCACCCTGCACCTCATTCCAACTCCTACCGCG
ATACAGACCCACAGAGTGCCATCCCTGAGAGACCAGACCGCTCCC
CAATACTCTCCTAAAATAAACATGAAGCACAAAAAAAAAAAAAAA
AAAAAAAAAAAAAAAAAAA

### Example 12: Treating Cancer

One or more cell engagers having the ability to bind to (a) a NKG2C polypeptide and (b) a CD33 polypeptide are administered to a human identified as having a leukemia (e.g., AML). The one or more cell engagers having the ability to bind to a NKG2C polypeptide are administered using intravenous injection. After the administration of one or more cell engagers having the ability to bind to a NKG2C polypeptide, the number of cancer cells within the human is reduced. After the administration of the one or more cell engagers having the ability to bind to a NKG2C polypeptide, the size of one or more tumors within the human is reduced.

### Example 13: Treating Cancer

One or more cell engagers having the ability to bind to (a) a NKG2C polypeptide and (b) a CD33 polypeptide and a population of NKG2C⁺ NK cells (e.g., as an adoptive cell therapy) are administered to a human identified as having a leukemia (e.g., AML). Both (1) the one or more cell engagers having the ability to bind to (a) a NKG2C polypeptide and (b) a CD33 polypeptide and (2) the population of NKG2C⁻ NK cells are administered using intravenous injection (e.g., as a single injection). After the administration of one or more cell engagers having the ability to bind to a NKG2C polypeptide and a CD33 polypeptide, the number of cancer cells within the human is reduced. After the administration of the one or more cell engagers having the ability to bind to a NKG2C polypeptide and a CD33 polypeptide, the size of one or more tumors within the human is reduced.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A cell engager comprising a first antigen binding domain and a second antigen binding domain, wherein said first antigen binding domain binds to a NKG2C polypeptide, and wherein said second antigen binding domain binds to a polypeptide expressed on the surface of a cancer cell.

2. The cell engager of claim 1, wherein said first antigen binding domain comprises a heavy chain variable domain comprising the amino acid sequences set forth in SEQ ID NO:5 (or SEQ ID NO:5 with one, two, or three amino acid additions, deletions, or substitutions), SEQ ID NO:6 (or SEQ ID NO:6 with one, two, or three amino acid additions, deletions, or substitutions), and SEQ ID NO:7 (or SEQ ID NO:7 with one, two, or three amino acid additions, deletions, or substitutions), and a light chain variable domain comprising the amino acid sequences set forth in SEQ ID NO:8 (or SEQ ID NO:8 with one, two, or three amino acid additions, deletions, or substitutions), SEQ ID NO:9 (or SEQ ID NO:9 with one, two, or three amino acid additions, deletions, or substitutions), and SEQ ID NO:10 (or SEQ ID NO:10 with one, two, or three amino acid additions, deletions, or substitutions).

3. The cell engager of claim 1, wherein said first antigen binding domain comprises (a) a heavy chain variable domain comprising the amino acid sequence set forth in SEQ ID NO:19 and (b) a light chain variable domain comprising the amino acid sequence set forth in SEQ ID NO:20.

4. The cell engager of claim 1, wherein said first antigen binding domain comprises a scFv.

5. The cell engager of claim 1, wherein said polypeptide expressed on the surface of said cancer cell is a CD33 polypeptide.

6. The cell engager of claim 1, wherein said second antigen binding domain comprises a heavy chain variable domain comprising the amino acid sequences set forth in SEQ ID NO:24 (or SEQ ID NO:24 with one, two, or three amino acid additions, deletions, or substitutions), SEQ ID NO:25 (or SEQ ID NO:25 with one, two, or three amino acid additions, deletions, or substitutions), and SEQ ID NO:26 (or SEQ ID NO:26 with one, two, or three amino acid additions, deletions, or substitutions), and a light chain variable domain comprising the amino acid sequences set forth in SEQ ID NO:27 (or SEQ ID NO:27 with one, two, or three amino acid additions, deletions, or substitutions), SEQ ID NO:28 (or SEQ ID NO:28 with one, two, or three amino acid additions, deletions, or substitutions), and SEQ ID NO:29 (or SEQ ID NO:29 with one, two, or three amino acid additions, deletions, or substitutions).

7. The cell engager of claim 1, wherein said second antigen binding domain comprises (a) a heavy chain variable domain comprising the amino acid sequence set forth in SEQ ID NO:38 and (b) a light chain variable domain comprising the amino acid sequence set forth in SEQ ID NO:39.

8. The cell engager of claim 1, wherein said second antigen binding domain comprises a scFv.

9. The cell engager of claim 1, wherein said cell engager comprises a linker located between said first antigen binding domain and said second antigen binding domain.

10. (canceled)

11. The cell engager of claim 1, wherein said cell engager further comprises an IL-15 polypeptide or a biologically active fragment of said IL-15 polypeptide.

12. The cell engager of claim 11, wherein said IL-15 polypeptide or said biologically active fragment of said IL-15 polypeptide is located between said first antigen binding domain and said second antigen binding domain.

13-14. (canceled)

15. A nucleic acid construct comprising a nucleic acid sequence encoding a cell engager comprising a first antigen binding domain and a second antigen binding domain, wherein said first antigen binding domain binds to a NKG2C polypeptide, and wherein said second antigen binding domain binds to a polypeptide expressed on the surface of a cancer cell.

16. The nucleic acid construct of claim 15, wherein said nucleic acid is a viral vector.

17. The nucleic acid construct of claim 15, wherein said nucleic acid is a phagemid.

18. A host cell comprising a nucleic acid comprising a nucleic acid sequence encoding a cell engager comprising a first antigen binding domain and a second antigen binding domain, wherein said first antigen binding domain binds to a NKG2C polypeptide, and wherein said second antigen binding domain binds to a polypeptide expressed on the surface of a cancer cell.

19. (canceled)

20. A method of treating a mammal having cancer, wherein said method comprises:
(a) administering, to said mammal, a cell engager comprising a first antigen binding domain and a second antigen binding domain, wherein said first antigen binding domain binds to a NKG2C polypeptide, and wherein said second antigen binding domain binds to a polypeptide expressed on the surface of a cancer cell, or
(b) administering said cell engager and a population of natural killer (NK) cells to said mammal.

21. The method of claim 20, wherein said mammal is a human.

22. The method of claim 20, wherein said cancer is a CD33⁺ cancer.

23. The method of claim 20, wherein said cancer is selected from the group consisting of a leukemia, a lymphoma, a myelodysplastic syndrome, and systemic mastocytosis.

24. The method of claim 20, wherein the number of cancer cells within said mammal is reduced following said administering step.

25-32. (canceled)
